(19) United States
(12) Patent Application Publication
(10) Pub. No.: US 2025/0228931 A1
(43) Pub. Date: Jul. 17, 2025

(54) ALPHAVIRUS REPLICON ENCODING CHIMERIC SARS-COV-2 RECEPTOR BINDING DOMAINS

(71) Applicant: VLP Therapeutics, Inc., Wilmington, DE (US)

(72) Inventors: Wataru AKAHATA, Kensington, MD (US); Jonathan F. SMITH, Redwood City, CA (US); Ryuji UENO, Easton, MD (US)

(73) Assignee: VLP Therapeutics, Inc., Wilmington, DE (US)

(21) Appl. No.: 19/098,884

(22) Filed: Apr. 2, 2025

Related U.S. Application Data
(63) Continuation of application No. 17/232,666, filed on Apr. 16, 2021, now Pat. No. 12,280,102.
(60) Provisional application No. 63/088,723, filed on Oct. 7, 2020.
(60) Provisional application No. 63/050,442, filed on Jul. 10, 2020.
(60) Provisional application No. 63/011,561, filed on Apr. 17, 2020.

(51) Int. Cl. A61K 39/215; C07K 14/165; C07K 14/18

(52) U.S. Cl. CPC A61K 39/215; C07K 14/165; C07K 14/1808; C07K 2319/02; C07K 2319/03; C12N 2770/20034; C12N 2770/36141

(57) ABSTRACT

Provided herein is an isolated polynucleotide, which encodes alphavirus non-structural proteins nsp1, nsp2, nsp3 and nsp4 and a polypeptide comprising a coronavirus protein fused to a signal sequence and/or transmembrane domain. The coronavirus protein may be the receptor binding domain of the S1 subunit of coronavirus spike (S) protein. The polynucleotide such as RNA is useful for as a vaccine against coronavirus infection, especially, COVID-19 infection.

### CROSS REFERENCE TO RELATED APPLICATIONS

This application is a Rule 53(b) Continuation of U.S. application Ser. No. 17/232,666, filed Apr. 16, 2021, which claims benefit of U.S. Provisional Patent Application Nos. 63/011,561 filed Apr. 17, 2020, 63/050,442 filed Jul. 10, 2020 and 63/088,723 filed Oct. 7, 2020, the disclosures of which are incorporated herein by reference in their entireties.

### INCORPORATION BY REFERENCE OF SEQUENCE LISTING

The content of the electronically submitted sequence listing, file name: Q307605_sequence listing as filed. XML; size 80,790 bytes; and date of creation of Mar. 21, 2025, filed herewith, is incorporated herein by reference in its entirety.

### TECHNICAL FIELD

The present disclosure relates generally to the field of a vaccine and to a method and a composition for treating and/or immunizing against viral infections. In particular, the present disclosure relates to a vaccine for coronavirus such as SARS-COV-2 (COVID-19).

### BACKGROUND

Coronaviruses are a large family of viruses that usually cause mild to moderate upper-respiratory tract illnesses, like the common cold. However, three new coronaviruses have emerged from animal reservoirs over the past two decades to cause serious and widespread illness and death.

There are hundreds of coronaviruses, most of which circulate among such animals as pigs, camels, bats and cats. Sometimes those viruses jump to humans—called a spillover event—and can cause disease. Four of the seven known coronaviruses that sicken people cause only mild to moderate disease. Three can cause more serious, even fatal, disease. SARS coronavirus (SARS-COV) emerged in November 2002 and caused severe acute respiratory syndrome (SARS). That virus disappeared by 2004. Middle East respiratory syndrome (MERS) is caused by the MERS coronavirus (MERS-COV). Transmitted from an animal reservoir in camels, MERS was identified in September 2012 and continues to cause sporadic and localized outbreaks. The third novel coronavirus to emerge in this century is called SARS-COV-2. It causes coronavirus disease 2019 (COVID-19), which emerged from China in December 2019 and was declared a global pandemic by the World Health Organization on Mar. 11, 2020. (niaid.nih.gov/diseases-conditions/coronaviruses)

Most people infected with the SARS-COV-2 virus will experience mild to moderate respiratory illness and recover without requiring special treatment. Older people, and those with underlying medical problems like cardiovascular disease, diabetes, chronic respiratory disease, and cancer are more likely to develop serious illness. At this time, there are no specific vaccines or treatments for COVID-19. (who.int/health-topics/coronavirus#tab=tab_1)

Because they are given to large numbers of healthy people, vaccines usually have a higher bar for safety than do drugs administered to people who are already ill. With SARS-COV-2 (COVID-19) vaccines, researchers' main safety concern is to avoid a phenomenon called disease enhancement, in which vaccinated people who do get infected develop a more severe form of the disease than people who have never been vaccinated. In studies of an experimental SARS vaccine reported in 2004, vaccinated ferrets developed damaging inflammation in their livers after being infected with the virus.

Antibody-dependent enhancement (ADE) is a mechanism by which some viruses such as dengue virus, feline coronavirus, and HIV, as an alternative strategy, can infect host cells that display Fc receptors and thereby take advantage of anti-viral humoral immune responses. Whether ADE occurs in SARS-Coronavirus infections is controversial, but it has been reported that antibodies against the spike protein of SARS-Coronavirus may cause ADE effect.

Peter Hotez, a vaccine scientist at Baylor College of Medicine in Houston, Texas, thinks potential vaccines should be tested in animals first to rule out disease enhancement, before trials move on to humans. He says he understands the reasoning for pushing SARS-COV-2 (COVID-19) vaccines to human tests quickly, but adds that, because of the possibility that a vaccine could enhance disease, “I'm not sure this is the vaccine you want to do it for”. (Non-Patent Literature 1)

Thus, there is a need in the art for a safer vaccine for coronaviruses that could avoid the ADE effect.

### CITATION LIST

Patent Literature 1: WO 2019/124441
Non-Patent Literature 1: Nature 579, 481 (2020)

The contents of the cited documents are herein incorporated by reference.

### SUMMARY OF THE INVENTION

The present disclosure relates to novel antigenically-active proteins/polypeptides capable of inducing protection against coronaviruses while minimizing the possibility of ADE. The protein/polypeptide disclosed herein include a coronavirus structural protein fused to a signal sequence and/or a transmembrane domain. The coronavirus structural protein may be a spike (S) protein, nucleocapsid (N) protein, membrane (M) protein, a small envelope protein (E) or a combination thereof. Specific examples may include S1 and/or S2 subunit of the spike protein and especially, receptor binding domain of the S1 subunit.

In another aspect, the present disclosure relates to a novel polynucleotide encoding the above discussed novel antigenically-active proteins/polypeptides capable of inducing protection against coronaviruses.

In another aspect, the present disclosure relates to a novel alphavirus replicon that can express the above discussed antigenically-active protein/polypeptide. The alphavirus replicon includes polynucleotide such as RNA encoding alphavirus non-structural proteins nsP1, nsP2, nsP3 and nsP4 and a polynucleotide encoding the above-discussed antigenically active protein/polypeptide as a gene of interest.

In yet another aspect, the present disclosure relates to a vaccine comprising the above discussed polypeptide or polynucleotide. Especially, the present disclosure provides a vaccine comprising a polynucleotide encoding alphavirus non-structural proteins nsP1, nsP2, nsP3 and nsP4, and a polypeptide comprising a coronavirus structural protein fused to a signal sequence and/or transmembrane domain. The vaccine can be used for preventing and/or treating a subject from coronavirus infection.

In yet another aspect, the present disclosure relates to a method for immunizing, preventing or treating a subject from coronavirus infection comprising administering an effective amount of the above-discussed polynucleotide or polypeptide to the subject in need thereof.

In still another aspect, the present disclosure relates to use of the above-discussed polypeptide or polynucleotide for the manufacture of a medicament.

### BRIEF DESCRIPTION OF THE DRAWINGS

FIG. 1 A construct of an alphavirus replicon.

FIG. 2 Full length VEEV TC-83 vector of the construct C including SS1-RBD-GS-IgG4-CH₃ (linker)-GS-TM1 produced in Example 3.

FIG. 3 Expression of RBD in HEK293T cells transfected with the alphavirus replicon plasmid vectors prepared in Example 3.

FIG. 4-1-1 and FIG. 4-1-2 Antibody titer against SARS-COV-2 Spike protein in mice sera on day 14 and day 35 immunization with the alphavirus replicon particles prepared in Example 4.

FIG. 4-2 Antibody titer against SARS-COV-2 Spike protein in mice sera on day 14 and day 35 immunization with the alphavirus replicon particles prepared in Example 4

FIG. 5-1-1 and FIG. 5-1-2 Antibody titer against SARS-COV-2 RBD in mice sera on day 14 and day 35 immunization with the alphavirus replicon particles prepared in Example 4.

FIG. 5-2 Antibody titer against SARS-COV-2 RBD in mice sera on day 14 and day 35 immunization with the alphavirus replicon particles prepared in Example 4.

FIG. 6 Antibody titer against SARS-COV-2 Spike S1 antigen in mice sera on day 7 immunization with the alphavirus replicon particles prepared in Example 4.

FIG. 7-1 and FIG. 7-2 Antibody titer against SARS-COV-2 S1 protein in mice sera on Day 14, 28, and 46 immunization with the alphavirus self-amplifying RNA prepared in Example 8.

### DETAILED DESCRIPTION OF THE INVENTION

As used herein “coronavirus” is meant to refer to single-stranded, positive-sense RNA viruses that belong to the family, Coronaviridae. Exemplary Coronaviridae viruses include but are not limited to SARS-COV, MERS-COV and SARS-COV-2 (COVID-19). SARS-COV-2 (COVID-19) may include known and unknown mutants. Known mutants may include SARS-COV-2 E484K_N501Y_K417T mutant (Brazil Strain Mutant), E484K_N501Y_K417N mutant (South African Mutant) and E484K mutant. The coronavirus genome encodes numerous non-structural proteins and four major structural proteins including the spike (S), nucleocapsid (N), membrane (M) and small envelope (E). Spike (S) protein, a large envelope glycoprotein, is composed of S1 and S2 subunits. The “Receptor-binding domain (RBD)” is located in the SI subunit. Preferable example of the structural protein used herein is coronavirus RBD. Spike protein, S1 and S2 subunits of the spike protein and RBD of SARS-COV-2 and its mutants have been identified and published (Wrapp et al., Science 10.1126/science.abb2507(2020), Z. Wang et al., (preprint) bioRxiv 2021.01.15.426911; doi: doi.org/10.1101/2021.01.15.426911).

“Coronavirus structural protein” used herein may be a naturally occurring virus structural protein or a modified protein thereof. The modified protein may be a fragment of the naturally occurring virus structural protein. In one embodiment, the modified protein has at least 70%, 75%, 80%, 85%, 90%, 95% or 98% amino acid sequence identity to a naturally occurring viral structural protein or its fragment. In one embodiment, the modified protein is a mutant where at most 10% of the amino acids are deleted, substituted, and/or added based on a naturally occurring viral envelope protein or its fragment.

As used herein, “transmembrane domain (TM)” is a protein derived either from a natural or from a synthetic source. Where the source is natural, the domain in some aspects is derived from any membrane-bound or transmembrane protein. In one aspect, the membrane-bound or transmembrane protein is a protein heterologous to SARS-COV-2. Examples of the membrane-bound or transmembrane proteins may include the alpha, beta or zeta chain of a T-cell receptor, CD28, CD3 epsilon, CD45, CD4, CD5, CDS, CD9, CD16, CD22, CD33, CD37, CD64, CD80, CD86, CD134, CD137, CD154; toll-like receptors (TLR) such as TLR1-TLR 10 in human and TLR1-TLR9, TLR11-TLR13 in mouse; interleukin (IL) receptors such as IL-1-28 receptor, RANTES receptors (CCR1, CCR3, CCR5), MIP-1 receptor, PF4 receptor, M-CSF receptor and NAP-2 receptor belonging to GPCR chemokine receptor; hemagglutinin (HA). In another aspect, the membrane-bound or transmembrane protein is a protein derived from SARS-COV-2, such as SARS-COV-2 spike protein.

Examples of transmembrane proteins may also include the followings: 5-Lipoxygenase-Activating Protein, ABC Transporters, ACBP, Amyloid beta (A4), Bcl-2 Inhibitors, BNIPs, CAAX protease, Cytochromes P450, E-NPPs, EPHA1, EPHA2, EPHA3, EPHA4, Fatty Acid Desaturases, Gamma secretase, Glucose transporter, Glycophorins, GPCR, HER2/ErbB2, HER3/ErbB3, HER4/ErbB4, HSD-11β, Hypoxia-induced Proteins, Immunoglobulins, Insulin receptor, Integrins, Ion channel, MAPEG, MFS, MinK Family, MPPs, Peptidase AD, Peptidase Family M48, Peptidase MA, Protein Jagged, Receptor-type Kinases, SNARE Complex, Sulfatases, TNF receptor, Transmembrane Proteins 14, Transporter, TROBP, VEGF receptors, Aldehyde Dehydrogenases, Ammonia and Urea transporters, FMN-linked Oxidoreductases, Leucine Rich Repeat (LRR)-Containing Transmembrane Proteins, Leukotriene C4 synthase, Lysosome-associated membrane glycoprotein, Major Intrinsic Protein (MIP)/FNT superfamily, Microsomal prostaglandin E synthase, N-(deoxy)ribosyltransferase-like Membrane Proteins, Neutral/alkaline Ceramidases, Oligosaccharyl Transferase, Pentameric Ligand-gated Ion Channels, Rhodopsin-like receptors and pumps, Single-helix ATPase Regulators, Squalene/phytoene Synthase, Stearoyl-CoA desaturase 1, Stannin (SNN) Membrane Proteins, T-cell Surface Glycoprotein CD3 Zeta Chain, Tetratricopeptide repeat (TPR) Alpha-Helical Repeat Proteins, Transmembrane Proteins with NAD(P)-binding Rossmann-fold Domains.

In addition, monotypic/peripheral proteins that attached to the lipid bilayer or other integral proteins and peptide may also be used as transmembrane proteins. Examples may include Alpha/Beta-Hydrolase, Annexins, Bet V1-Like Protein, C1 Domain-Containing Protein, C2 Domain-containing Protein, CoA-Dependent Acyltransferases, CRAL-TRIO Domain-Containing Protein, DNase I-like protein, Fibrinogen, FYVE/PHD Zinc Finger Protein, Galactose-Binding Domain-Like Protein, Glycolipid Transfer Protein, Immunoglobulin-Like Superfamily (E Set) Protein, Lipocalin, Lipoxygenase, PGBD superfamily, PH Domain-Like Protein, Phosphatidylinositol 3-/4-Kinase, PLC-like Phosphodiesterase, Phosphotyrosine Protein Phosphatases II, P-Loop Containing Nucleoside Triphosphate Hydrolase, Protein kinase superfamily, PX Domain-Containing Protein, Saposin, Synuclein and Transcriptional factor tubby.

The expression “transmembrane domain” used in the present disclosure includes at least transmembrane region(s) of the membrane-bound or transmembrane protein. In addition, the transmembrane domain may also include juxtamembrane domain (JMD) and/or cytoplasmic tail of the membrane-bound or transmembrane protein.

Alternatively, the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain.

Preferable transmembrane domain may be those derived from influenza virus hemagglutinin (HA), CD80, Toll-like receptor 4 (TLR4) or SARS-COV-2 spike protein. Specific examples may include a protein consisting of the flexible juxtamembrane region or flexible linker, the transmembrane domain and the cytoplasmic tail of Influenza virus hemagglutinin “HA (flexible-TM-Cyt)”; a protein consisting of transmembrane domain and cytoplasmic tail of human CD80; a protein consisting of transmembrane domain (TM) and Toll/interleukin-1 receptor domain (TIR), and a protein consisting of the juxtamembrane domain (JMD) and TM of SARS-COV-2 Spike (S).

As used herein, “signal sequence” (sometimes referred to as signal peptide, targeting signal, localization signal, localization sequence, transit peptide, leader sequence or leader peptide) is a polynucleotide or polypeptide, depending on the context. Signal sequence is from about 9 to 200 nucleotides or 3-70 amino acids in length that, optionally, is incorporated at the 5′ or N-terminus of the coding region or the protein. Some signal sequences are cleaved from the protein, for example by a signal peptidase after the proteins are transported to the desired site.

In some embodiments, the signal sequence of IL-2, especially human IL-2 may be employed. In another embodiments, the signal sequence of the SARS-COV-2 spike protein may be employed

The coronavirus structural protein, the transmembrane domain and/or the signal sequence may be directly or indirectly fused. In one embodiment, one or two linkers may intervene between them.

Also the coronavirus structural protein, the transmembrane domain and/or the signal sequence can be truncated and replaced by short linkers. In some embodiments, the coronavirus structural protein, the transmembrane domain and/or the signal sequence include one or more peptide linkers.

An example of a short linker consists of from 2 to 25 amino acids (e.g. 2, 3, 4, 5 or 6 amino acids). Usually, it is from 2 to 15 amino acids in length, such as SG, GS, SGG, GGS SGSG and TRGGS. In certain circumstances, the linker can consist of only one amino acid, such as glycine (G), serine (S) and cysteine (C).

When the coronavirus structural protein is chemically conjugated to the transmembrane domain and/or the signal sequence through a chemical cross-linker, examples of the cross-linkers include, but are not limited to, SMPH, Sulfo-MBS, Sulfo-EMCS, Sulfo-GMBS, Sulfo-SIAB, Sulfo-SMPB, Sulfo-SMCC, SVSB, and SIA. Chemical cross-linkers available on the market may also be employed.

IgG-derived substances can also be used as a linker. Examples of IgG-derived substances include IgG1 to IgG4 comprising (i) full (hinge-CH₂CH₃) (ii) half (hinge-CH₃) and (iii) short (12aa hinge only). Preferable example is IgG4-CH₃.

As used herein “alphavirus” is meant to refer to RNA-containing viruses that belong to the Togaviridae family of viruses. Exemplary Togaviridae viruses include but are not limited to Eastern Equine Encephalitis Virus (EEEV), Venezuelan Equine Encephalitis Virus (VEEV), Everglades Virus, Mucambo Virus, Pixuna Virus, Western Equine Encephalitis Virus (WEEV), Sindbis Virus, Semliki Forest Virus, Middleburg Virus, Chikungunya Virus (CHIKV), O'nyong-nyong Virus, Ross River Virus, Barmah Forest Virus, Getah Virus, Sagiyama Virus, Bebaru Virus, Mayaro Virus, Una Virus, Aura Virus, Whataroa Virus, Babanki Virus, Kyzylagach Virus, Highlands J virus, Fort Morgan Virus, Ndumu Virus, Buggy Creek Virus and Ockelbo virus.

By “alphavirus structural protein” is meant a polypeptide or fragment thereof having at least about 80% amino acid sequence identity to a naturally occurring viral capsid or envelope protein. In one embodiment, the alphavirus structural protein bas at least about 85%, 90%, 95% or greater amino acid sequence identity with Eastern Equine Encephalitis Virus (EEEV), Venezuelan Equine Encephalitis Virus (VEEV), Everglades Virus, Mucambo Virus, Pixuna Virus, Western Equine Encephalitis Virus (WEEV), Sindbis Virus, Semliki Forest Virus, Middleburg Virus, Chikungunya Virus (CHIKV), O'nyong-nyong Virus, Ross River Virus, Barmah Forest Virus, Getah Virus, Sagiyama Virus, Bebaru Virus, Mayaro Virus, Una Virus, Aura Virus, Whataroa Virus, Babanki Virus, Kyzylagach Virus, Highlands J virus, Fort Morgan Virus, Ndumu Virus, or Buggy Creek Virus. Wild type amino acid sequences of alphavirus structural proteins can be obtained from GenBank.

In specific embodiments, the alphavirus is a CHIKV, for example CHIKV strain 37997 or LR2006 OPY-1. In other embodiments, the alphavirus is a VEEV, for example VEEV strain TC-83.

By “an alphavirus replicon” is meant an RNA molecule which can direct its own amplification in vivo in a target cell. The replicon encodes the polymerase(s) which catalyze RNA amplification (nsP1, nsP2, nsP3, nsP4) and contains cis RNA sequences required for replication which are recognized and utilized by the encoded polymerase(s). An alphavirus replicon typically contains the following ordered elements: 5′ UTR, sequences which encode alphavirus nonstructural proteins (nsP1, nsP2, nsP3, nsP4), 3′ UTR, and a poly A signal. An alphavirus replicon also contains one or more viral sub-genomic promoters directing the expression of the gene of interest. Those sequences may have one or more mutations taught in a prior art.

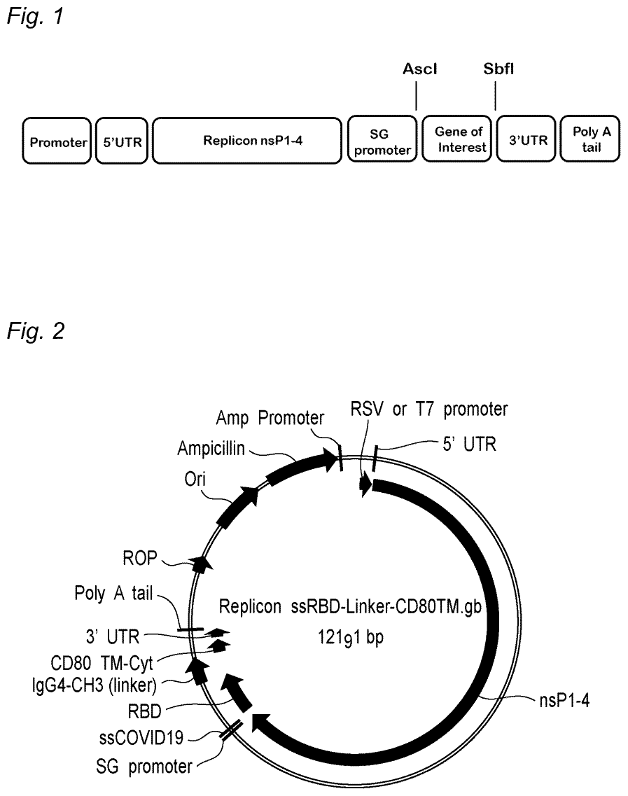

The alphavirus replicon provided by the present disclosure may have the construct shown in FIG. 1.

In this disclosure, “comprises,” “comprising,” “containing” and “having” and the like can have the meaning ascribed to them in U.S. patent law and can mean “includes,” “including,” and the like; “consisting essentially of or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or there between.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e<″3> and e<″100> indicating a closely related sequence.

By “effective amount” is meant the amount of an agent required to ameliorate the symptoms of a disease relative to an untreated patient. The effective amount of active compound(s) used to practice the present invention for prevention or treatment of a disease varies depending upon the manner of administration, the age, body weight, and general health of the subject. Ultimately, the attending physician or veterinarian will decide the appropriate amount and dosage regimen. Such amount is referred to as an “effective” amount.

A satisfactory effect can be obtained by systemic administration, e.g. intramuscular administration, subcutaneous administration or intravenous administration 1-4 times at the amount of 10³-10¹⁰ Infectious Unit (IU) or 0.01-500 μg per time, preferably 10⁵-10¹⁰ IU or 0.1-100 μg per time, for example 10⁷-10⁹ IU or 1-50 μg per one time. The replicon may preferably be formulated in a vaccine composition suitable for administration in a conventional manner.

By “subject” is meant a mammal, including, but not limited to, a human or non-human mammal, such as a bovine, equine, canine, ovine, or feline.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

Unless specifically stated or obvious from context, as used herein, the term “or” is understood to be inclusive.

Unless specifically stated or obvious from context, as used herein, the terms “a”, “an”, and “the” are understood to be singular or plural.

The art will acknowledge that polynucleotide sequences described in the specification and claims will recite “T”s in a representative DNA sequence but where the sequence represents RNA, the “T”'s would be substituted for “U”s.

Any vaccine compositions or methods provided herein can be combined with one or more of any of the other vaccine compositions and methods provided herein.

The term “vector” refers to the means by which a nucleic acid sequence can be propagated and/or transferred between organisms, cells, or cellular components. Vectors include plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating. In many, but not all, common embodiments, the vectors of the present invention are plasmids or bacmids.

Typically, the nucleic acid molecule to be expressed is “operably linked” to a promoter and/or enhancer, and is subject to transcription regulatory control by the promoter and/or enhancer.

The method of transfection and the choice of expression vehicle will depend on the host system selected. Transfection methods are described, e.g., in Ausubel et al. (supra); expression vehicles may be chosen from those provided, e.g., in Cloning Vectors: A Laboratory Manual (P. H. Pouwels et al., 1985, Supp. 1987) The references cited in this paragraph are herein incorporated by reference.

A variety of expression systems exist for the production of the constructs of the invention. Expression vectors useful for producing the constructs include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as alphavirus (e.g. Chikungunya Virus (CHIKV) and Venezuelan Equine Encephalitis Virus (VEEV)), baculoviruses, papova viruses, such as SV40, vaccinia viruses, adenoviruses, fowl pox viruses, pseudorabies viruses and retroviruses, and vectors derived from combinations thereof.

Constructs and/or vectors used herein comprise alphavirus polynucleotides encoding nonstructural proteins nsP1, nsP2, nsP3 and nsP4 and a gene of interest encoding a polypeptide comprising a coronavirus structural protein fused to a signal sequence and/or a transmembrane domain as discussed above. Specific example of the construct or vector is that shown in FIG. 1.

The vector may be, for example, a phage, plasmid, viral, or retroviral vector. The constructs and/or vectors that comprise the nucleotides should be operatively linked to an appropriate promoter, such as the CMV promoter, phage lambda PL promoter, the E. coli lac, phoA and tac promoters, the SV40 early and late promoters, and promoters of retroviral LTRs are non-limiting examples. Other suitable promoters will be known to the skilled artisan depending on the host cell and/or the rate of expression desired. The expression constructs will further contain sites for transcription initiation, termination, and, in the transcribed region, a ribosome-binding site for translation. The coding portion of the transcripts expressed by the constructs will preferably include a translation initiating codon at the beginning and a termination codon appropriately positioned at the end of the polypeptide to be translated.

Vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Among vectors preferred are virus vectors, such as baculovirus, poxvirus (e.g., vaccinia virus, avipox virus, canarypox virus, fowlpox virus, raccoonpox virus, swinepox virus, etc.), adenovirus (e.g., canine adenovirus), herpesvirus, and retrovirus. Other vectors that can be used with the invention comprise vectors for use in bacteria, which comprise pQE70, pQE60 and pQE-9, pBluescript vectors, Phagescript vectors, pNH8A, pNH16a, pNH18A, pNH46A, ptrc99a, pKK223-3, pKK233-3, pDR540, pRIT5. Among preferred eukaryotic vectors are pFastBacl pWINEO, pSV2CAT, pOG44, pXT1 and pSG, pSVK3, pBPV, pMSG, and pSVL. Other suitable vectors will be readily apparent to the skilled artisan.

Recombinant constructs can be prepared and used to transfect, can express viral proteins, including those described herein, into eukaryotic cells and/or prokaryotic cells. Thus, in one embodiment, the present disclosure provides host cells which comprise a vector (or vectors) that contain nucleic acids which encode alphavirus structural proteins, including capsid, E3, E2, 6K, and E1 or portions thereof, and a vector that comprises nucleic acids which encode alphavirus nsP1, nsP2, nsP3 and nsP4, and at least one of coronavirus gene of interest under conditions which allow the formation of alphavirus replicon particle.

In one embodiment, said vector is a recombinant baculovirus. In another embodiment, said recombinant baculovirus is transfected into an insect cell. In a preferred embodiment, said cell is an insect cell. In another embodiment, said insect cell is a Sf9 cell.

One particular bacterial expression system for polypeptide production is the E. coli pET expression system (Novagen, Inc., Madison, Wis). According to this expression system, DNA encoding a polypeptide is inserted into a pET vector in an orientation designed to allow expression. Since the gene encoding such a polypeptide is under the control of the T7 regulatory signals, expression of the polypeptide is achieved by inducing the expression of T7 RNA polymerase in the host cell. This is typically achieved by using host strains that express T7 RNA polymerase in response to IPTG induction. Once produced, a recombinant polypeptide is then isolated according to standard methods known in the art, for example, those described herein.

Depending on the vectors and host cells selected, the constructs are produced by growing host cells transfected by the vectors under conditions whereby the recombinant proteins arc expressed and the alphavirus replicon is generated, and constructs containing alphavirus replicon being packaged with the particle of alphavirus structural proteins are formed. In one embodiment, the invention comprises a method of producing a construct, that involves co-transfecting a vector comprising a polynucleotide encoding alphavirus non-structural protein nsP1, nsP2, nsP3 and nsP4, and at least one gene of interest encoding the polypeptide comprising a coronavirus structural protein fused to a signal sequence and/or transmembrane domain, and at least one vector each encoding at least one alphavirus structural protein into suitable host cells and expressing said alphavirus structural protein under conditions that allow construct formation. In another embodiment, the eukaryotic cell is selected from the group consisting of, yeast, insect, amphibian, avian or mammalian cells. The selection of the appropriate growth conditions is within the skill or a person with skill of one of ordinary skill in the art.

Methods to grow cells that produce alphavirus replicon particles of the invention include, but are not limited to, batch, batch-fed, continuous and perfusion cell culture techniques. In one embodiment, cells co-transfected with a vector encoding an alphavirus replicon and a vector comprising a polypeptide encoding capsid, and a vector comprising a polynucleotide encoding envelope proteins, such as those derived from a CHIKV or VEEV are grown in a bioreactor or fermentation chamber where cells propagate and express protein (e.g., recombinant proteins) for purification and isolation. Typically, cell culture is performed under sterile, controlled temperature and atmospheric conditions. A bioreactor is a chamber used to culture cells in which environmental conditions such as temperature, atmosphere, agitation and/or pH can be monitored. In one embodiment, the bioreactor is a stainless steel chamber. In another embodiment, said bioreactor is a pre-sterilized plastic bag (e.g., Cellbag®, Wave Biotech, Bridgewater, N.J., the contents of the cited document is herein incorporated by reference). In other embodiment, said pre-sterilized plastic bags are about 10 L to 1000 L bags.

In another embodiment, an RNA molecule such as an alphavirus replicon may be generated by conventional procedures known to the art from a template DNA sequence. In vitro transcription (IVT) methods permit template-directed synthesis of RNA molecules. IVT methods permit synthesis of large quantities of RNA transcript. Generally, IVT utilizes a DNA template comprising a promoter sequence upstream of a sequence of interest. The promoter sequence is most commonly of bacteriophage origin such as the T7, T3 or SP6 promoter sequence but many other promotor sequences can be tolerated including those designed de novo. Transcription of the DNA template is typically best achieved by using the RNA polymerase corresponding to the specific bacteriophage promoter sequence. Exemplary RNA polymerases include, but are not limited to T7 RNA polymerase, T3 RNA polymerase, or SP6 RNA polymerase, among others. IVT is generally initiated at a dsDNA but can proceed on a single strand. Kits for in vitro transcription such as T7 transcription kit (RiboMax™ Express Large Scale RNA production System, Promega (WI USA)).

As used herein, the term “pharmaceutically acceptable carrier” means one or more compatible solid or liquid fillers, diluents or encapsulating substances which are suitable for administration to a human or other vertebrate animal, including any and all aqueous solvents (e.g., water, alcoholic/aqueous solutions, saline solutions, parenteral vehicles, such as sodium chloride, and Ringer's dextrose), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a vaccine composition are adjusted according to well-known parameters.

Encapsulating substances refers to a delivery vehicle where the polynucleotide or vector is packaged, such as a replicon particle (e.g. the alphavirus replicon particle described in US patent publication No. 2019-0185822, the contents of the document is incorporated by reference) and a lipid delivery system (e.g. liposome).

In some embodiments, the vaccine compositions or formulations of the present disclosure comprise a lipid delivery system, e.g., a liposome, a lipoplexes, a lipid nanoparticle, or any combination thereof. The polynucleotides such as an alpha virus replicon described herein can be formulated using one or more liposomes, lipoplexes, or lipid nanoparticles. Liposomes, lipoplexes, or lipid nanoparticles can be used to improve the efficacy of the polynucleotides directed protein production as these formulations can increase cell transfection by the polynucleotide; and/or increase the translation of encoded protein. The liposomes, lipoplexes, or lipid nanoparticles can also be used to increase the stability of the polynucleotides.

Liposomes are artificially-prepared vesicles that may primarily be composed of a lipid bilayer and may be used as a delivery vehicle for the administration of pharmaceutical formulations. Liposomes can be of different sizes. A multilamellar vesicle (MLV) may be hundreds of nanometers in diameter, and may contain a series of concentric bilayers separated by narrow aqueous compartments. A small unicellular vesicle (SUV) may be smaller than 50 nm in diameter, and a large unilamellar vesicle (LUV) may be between 50 and 500 nm in diameter. Liposome design may include, but is not limited to, opsonins or ligands to improve the attachment of liposomes to unhealthy tissue or to activate events such as, but not limited to, endocytosis. Liposomes may contain a low or a high pH value in order to improve the delivery of the pharmaceutical formulations.

The formation of liposomes may depend on the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimal size, polydispersity and the shelf-life of the vesicles for the intended application, and the batch-to-batch reproducibility and scale up production of safe and efficient liposomal products, etc.

In some embodiments, the polynucleotides such as alpha virus replicon described herein may be encapsulated by the liposome and/or it may be contained in an aqueous core that may then be encapsulated by the liposome.

In some embodiments, the polynucleotides such as alpha virus replicon described herein can be formulated in a cationic oil-in-water emulsion where the emulsion particle comprises an oil core and a cationic lipid that can interact with the polynucleotide anchoring the molecule to the emulsion particle. In some embodiments, the polynucleotides described herein can be formulated in a water-in-oil emulsion comprising a continuous hydrophobic phase in which the hydrophilic phase is dispersed.

In some embodiments, the polynucleotides such as alpha virus replicon described herein can be formulated in a lipid-polycation complex. As a non-limiting example, the polycation can include a cationic peptide or a polypeptide such as, but not limited to, polylysine, polyornithine and/or polyarginine and the cationic peptides.

In some embodiments, the polynucleotides such as alpha virus replicon described herein can be formulated in a lipid nanoparticle (LNP).

Lipid nanoparticle formulations typically comprise one or more lipids. In some embodiments, the lipid is a cationic or an ionizable lipid. In some embodiments, lipid nanoparticle formulations further comprise other components, including a phospholipid, a structural lipid, a quaternary amine compound, and a molecule capable of reducing particle aggregation, for example a PEG or PEG-modified lipid. In some embodiments, the amount of the cationic and ionizable lipids in the lipid composition ranges from about 0.01 mol % to about 99 mol %.

LNPs contain a pH-sensitive ionizable cationic lipid that attract anionic nucleic acids to form the core of self-assembling nanoparticle to ensure high encapsulation. At physiological pH, LNPs are neutral, eliminating a mechanism of toxicity seen with permanently cationic molecules.

These same pH-sensitive lipids are responsible for responding to the acidic environment of the endosome and triggering the disruption of the endosome and release of the nucleic acid into the cell.

This replicon based vaccine technology is a unique platform technology for the vaccination as a RNA can self-amplify to produce the vaccine antigen and deliver into the cellular organ. Moreover, this replicon based vaccine technology overcomes the challenges commonly associated with DNA based vaccines, such as risk of genome integration or the high doses and devices needed for administration, e.g. electroporation, and expects the higher immunogenicity with minimum dose based on the self-replication system over the mRNA technology.

According to the present invention, novel antigenically-active proteins/polypeptides are also useful for producing antibodies for diagnosis and protecting against coronaviruses while minimizing the possibility of ADE. The proteins/polypeptides disclosed herein include minimum sequences encoding the coronavirus RBD fused to a signal sequence and/or to a transmembrane domain (TMD) sequence, intended to maximize immunogenicity and minimize ADE.

The invention will be described in detail with reference to the following examples, which, however, are not intended to limit the scope of the present application.

### EXAMPLES

### Example 1

Each gene encoding shown below construct 1-8 was synthesized by Integrated DNA Technologies, Inc. (idtdna.com/pages).

1. Construct of SARS-COV-2-RBD Sequence Fused to hIL-2 Signal Sequence and HA (Flexible Domain-Transmembrane(TM)-Cytoplasmic Tail(Cyt))

Construct 1

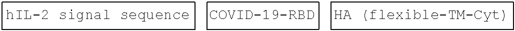

MYRMQLLSCIALSLALVTNSVRFPNITNLCPFGEVENATRFASVYAWNRKRISNC
VADYSVLYNSASFSTFKCYGVSPTKLNDLCFTNVYADSFVIRGDEVRQIAPGQTG
KIADYNYKLPDDFTGCVIAWNSNNLDSKVGGNYNYLYRLFRKSNLKPFERDISTE
IYQAGSTPCNGVEGFNCYFPLQSYGFQPTNGVGYQPYRVVVLSFELLHAPATVCG
PKKSGVKLESMGIYQILAIYSTVASSLVLLVSLGAISFWMCSNGSLQCRICI
(SEQ ID NO: 1)
hIL-2 signal sequence:
MYRMQLLSCIALSLALVINS (SEQ ID NO: 2)
COVID19-RBD:
VRFPNITNLCPFGEVENATRFASVYAWNRKRISNCVADYSVLYNSASFSTFKCYG
VSPTKLNDLCFTNVYADSFVIRGDEVRQIAPGQTGKIADYNYKLPDDFTGCVIAW
NSNNLDSKVGGNYNYLYRLFRKSNLKPFERDISTEIYQAGSTPCNGVEGENCYFP
LQSYGFQPTNGVGYQPYRVVVLSFELLHAPATVCGPKKS (SEQ ID NO: 3)
HA(flexible-TM-Cyt):
GVKLESMGIYQILAIYSTVASSLVLLVSLGAISFWMC
SNGSLQCRICI (SEQ ID NO: 4)

hIL-2: human Interleukin-2
HA(flexible-TM-Cyt): polypeptide of Influenza virus
   2. Construct of SARS-COV-2-RBD Sequence Fused to hIL-2 Signal Sequence and HA (Flexible-TM-Cyt)

Construct 2

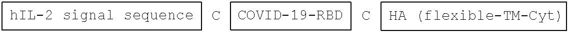

MYRMQLLSCIALSLALVTNSCVRFPNITNLCPFGEVFNATRFASVYAWNRKRISNCVA
DYSVLYNSASFSTFKCYGVSPTKLNDLCFTNVYADSFVIRGDEVRQIAPGQTGKIADY
NYKLPDDFTGCVIAWNSNNLDSKVGGNYNYLYRLFRKSNLKPFERDISTEIYQAGSTP
CNGVEGFNCYFPLQSYGFQPTNGVGYQPYRVVVLSFELLHAPATVCGPKKSCGVKLE
SMGIYQILAIYSTVASSLVLLVSLGAISFWMCSNGSLQCRICI (SEQ ID NO: 5)

“C” in the schematic chart: linker (Cysteine)

Linkers in the amino acid sequence are underlined.

3. Construct of SARS-COV-2-RBD Sequence Fused to hIL-2 Signal Sequence and HA (Flexible-TM-Cyt) With Linker

Construct 3

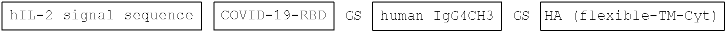

MYRMQLLSCIALSLALVTNSVRFPNITNLCPFGEVFNATRFASVYAWNRKRISNCVADYSVLYNSASFSTFKCYGVS
PTKLNDLCFTNVYADSFVIRGDEVRQIAPGQTGKIADYNYKLPDDFTGCVIAWNSNNLDSKVGGNYNYLYRLFRKS
NLKPFERDISTEIYQAGSTPCNGVEGFNCYFPLQSYGFQPTNGVGYQPYRVVVLSFELLHAPATVCGPKKSGSGQPR
EPQVYTLPPSQEEMTKNQVSLTCLVKGFYPSDIAVEWESNGQPENNYKTTPPVLDSDGSFFLYSRLTVDKSRWQEG
NVFSCSVMHEALHNHYTQKSLSLSLGKGSGVKLESMGIYQILAIYSTVASSLVLLVSLGAISFWMCSNGSLQCRICI
(SEQ ID NO: 6)

“GS” in the schematic chart: linker (GGATCC)

linkers are underlined in the amino acid sequence

human IgG4 CH3:
(SEQ ID NO: 7)
GQPREPQVYTLPPSQEEMTKNQVSLTCLVKGFYPSDIAVEWESNGQPEN
NYKTTPPVLDSDGSFFLYSRLTVDKSRWQEGNVFSCSVMHEALHNHYTQ
KSLSLSLGK

4. Construct of SARS-COV-2-RBD Sequence Fused to hIL-2 Signal Sequence and HA (Flexible-TM-Cyt) With Linker

Construct 4

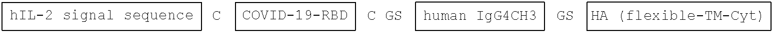

MYRMQLLSCIALSLALVTNSCVRFPNITNLCPFGEVFNATRFASVYAWNRKRISNCVADYSVLYNSASFSTFKCYGVSPTKL
NDLCFTNVYADSFVIRGDEVRQIAPGQTGKIADYNYKLPDDFTGCVIAWNSNNLDSKVGGNYNYLYRLFRKSNLKPFERDI
STEIYQAGSTPCNGVEGFNCYFPLQSYGFQPTNGVGYQPYRVVVLSFELLHAPATVCGPKKSCGSGQPREPQVYTLPPSQEE
MTKNQVSLTCLVKGFYPSDIAVEWESNGQPENNYKTTPPVLDSDGSFFLYSRLTVDKSRWQEGNVFSCSVMHEALHNHYT
QKSLSLSLGKGSGVKLESMGIYQILAIYSTVASSLVLLVSLGAISFWMCSNGSLQCRICI (SEQ ID NO: 8)

“C” “CGS” and “GS” in the schematic chart: linker

Linkers are underlined in the amino acid sequence.

5. Construct of SARS-COV-2-RBD Sequence Fused to hIL-2 Signal Sequence and Human TLR4 (TM-Toll/Interleukin-1 Receptor Domain (TIR))

Construct 5

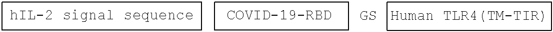

MYRMQLLSCIALSLALVTNSVRFPNITNLCPFGEVFNATRFASVYAWNRKRISNCVAD
YSVLYNSASFSTFKCYGVSPTKLNDLCFTNVYADSFVIRGDEVRQIAPGQTGKIADYN
YKLPDDFTGCVIAWNSNNLDSKVGGNYNYLYRLFRKSNLKPFERDISTEIYQAGSTPC
NGVEGFNCYFPLQSYGFQPTNGVGYQPYRVVVLSFELLHAPATVCGPKKSGSKTIIGV
SVLSVLVVSVVAVLVYKFYFHLMLLAGCIKYGRGENIYDAFVIYSSQDEDWVRNELV
KNLEEGVPPFQLCLHYRDFIPGVAIAANIIHEGFHKSRKVIVVVSQHFIQSRWCIFEYEI
AQTWQFLSSRAGIIFIVLQKVEKTLLRQQVELYRLLSRNTYLEWEDSVLGRHIFWRRL
RKALLDGKSWNPEGTVGTGCNWQEATSI (SEQ ID NO: 9)
human TLR4(TM-TIR):
KTIIGVSVLSVLVVSVVAVLVYKFYFHLMLLAGCIKYGRGENIYDAFVIYSSQDEDW
VRNELVKNLEEGVPPFQLCLHYRDFIPGVAIAANIIHEGFHKSRKVIVVVSQHFIQSR
WCIFEYEIAQTWQFLSSRAGIIFIVLQKVEKTLLRQQVELYRLLSRNTYLEWEDSVLG
RHIFWRRLRKALLDGKSWNPEGTVGTGCNWQEATSI (SEQ ID NO: 10)

6. Construct of SARS-COV-2-RBD Gene Sequence Fused to hIL-2 Signal Sequence and Human TLR4 (TM-TIR)

Construct

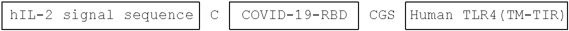

MYRMQLLSCIALSLALVTNSCVRFPNITNLCPFGEVFNATRFASVYAWNRKRISNCVADYSVLYN
SASFSTFKCYGVSPTKLNDLCFTNVYADSFVIRGDEVRQIAPGQTGKIADYNYKLPDDFTGCVIAW
NSNNLDSKVGGNYNYLYRLFRKSNLKPFERDISTEIYQAGSTPCNGVEGFNCYFPLQSYGFQPTNG
VGYQPYRVVVLSFELLHAPATVCGPKKSCGSKTIIGVSVLSVLVVSVVAVLVYKFYFHLMLLAGC
IKYGRGENIYDAFVIYSSQDEDWVRNELVKNLEEGVPPFQLCLHYRDFIPGVAIAANIIHEGFHKSR
KVIVVVSQHFIQSRWCIFEYEIAQTWQFLSSRAGIIFIVLQKVEKTLLRQQVELYRLLSRNTYLEWED
SVLGRHIFWRRLRKALLDGKSWNPEGTVGTGCNWQEATSI (SEQ ID NO: 11)

“C” and “CGS” in the schematic chart: linkers

Linkers are underlined in the amino acid sequence.

7. Construct of SARS-COV-2-RBD Sequence Fused to hIL-2 Signal Sequence and Human TLR4 (TM-TIR) With Linker

Construct 7

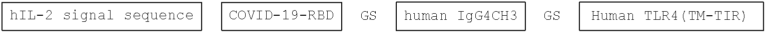

MYRMQLLSCIALSLALVTNSVRFPNITNLCPFGEVENATRFASVYAWNRKRISNCVADYSVLYNSASFSTFKCYGVSPTKL
NDLCFTNVYADSFVIRGDEVRQIAPGQTGKIADYNYKLPDDFTGCVIAWNSNNLDSKVGGNYNYLYRLFRKSNLKPFER
DISTEIYQAGSTPCNGVEGENCYFPLQSYGFQPTNGVGYQPYRVVVLSFELLHAPATVCGPKKSGSGQPREPQVYTLPPSQ
EEMTKNQVSLTCLVKGFYPSDIAVEWESNGQPENNYKTTPPVLDSDGSFFLYSRLTVDKSRWQEGNVFSCSVMHEALHN
HYTQKSLSLSLGKGSKTIIGVSVLSVLVVSVVAVLVYKFYFHLMLLAGCIKYGRGENIYDAFVIYSSQDEDWVRNELVKN
LEEGVPPFQLCLHYRDFIPGVAIAANIIHEGFHKSRKVIVVVSQHFIQSRWCIFEYEIAQTWQFLSSRAGIIFIVLQKVEKTLL
RQQVELYRLLSRNTYLEWEDSVLGRHIFWRRLRKALLDGKSWNPEGTVGTGCNWQEATSI (SEQ ID NO: 12)

“GS” in the schematic chart: linker

Linkers are underlined in the amino acid sequence.

8. Construct of COVID19 RBD Sequence Fused to hIL-2 Signal Sequence and Human TLR4 (TM-TIR) With Linker

Construct 8

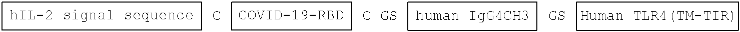

MYRMQLLSCIALSLALVTNSCVRFPNITNLCPFGEVENATRFASVYAWNRKRISNCVADYSVLYNSASFSTFKCYG
VSPTKLNDLCFTNVYADSFVIRGDEVRQIAPGQTGKIADYNYKLPDDFTGCVIAWNSNNLDSKVGGNYNYLYRLF
RKSNLKPFERDISTEIYQAGSTPCNGVEGFNCYFPLQSYGFQPTNGVGYQPYRVVVLSFELLHAPATVCGPKKSCG
SGQPREPQVYTLPPSQEEMTKNQVSLTCLVKGFYPSDIAVEWESNGQPENNYKTTPPVLDSDGSFFLYSRLTVDKS
RWQEGNVFSCSVMHEALHNHYTQKSLSLSLGKGSKTIIGVSVLSVLVVSVVAVLVYKFYFHLMLLAGCIKYGRGE
NIYDAFVIYSSQDEDWVRNELVKNLEEGVPPFQLCLHYRDFIPGVAIAANIIHEGFHKSRKVIVVVSQHFIQSRWCIF
EYEIAQTWQFLSSRAGIIFIVLQKVEKTLLRQQVELYRLLSRNTYLEWEDSVLGRHIFWRRLRKALLDGKSWNPEG
TVGTGCNWQEATSI (SEQ ID NO: 13)

“C, “CGS” and “GS” in the schematic chart: linker

Linkers are underlined in the amino acid sequence.

Information regarding hIL-2 signal sequence, COVID19-RBD, human IgG4 CH3 and HA(flexible-TM-Cyt) are also available from the National Center for Biotechnology Information website and from UniProt website.

hIL-2 Signal Sequence
ncbi.nlm.nih.gov/protein/P60568
UniProtKB/Swiss-Prot: P60568.1

### COVID19-RBD

ncbi.nlm.nih.gov/nuccore/NC_045512
NCBI Reference Sequence: NC_045512.2

### HA(flexible-TM-Cyt)

ncbi.nlm.nih.gov/protein/P03452
UniProtKB/Swiss-Prot: P03452.2

### Human IgG4

uniprot.org/uniprot/P01861
UniProtKB: P01861

### Example 2

Each gene encoding shown below construct A-O was synthesized by Integrated DNA Technologies, Inc. (idtdna.com/pages).

[symbol]

- [symbol] - [symbol] -

[symbol]

Construct A: SS1-RBD1(330-521) (SEQ ID NO: 37)
Construct B: SS2-RBD1(330-521)-TM2 (SEQ ID NO: 38)
Construct C: SS1-RBD1(330-521)-GS-Linker-GS-TM1 (SEQ ID NO: 39)
Construct D: SS1-RBD1(330-521)-GS-Linker-GS-TM2 (SEQ ID NO: 40)
Construct E: SS1-RBD2(330-530)-GS-Linker-GS-TM1 (SEQ ID NO: 41)
Construct F: SS1-RBD2(330-530)-GS-Linker-GS-TM2 (SEQ ID NO: 42)
Construct G: SS2-RBD1(330-521)-GS-Linker-GS-TM1 (SEQ ID NO: 43)
Construct H: SS2-RBD1(330-521)-GS-Linker-GS-TM2 (SEQ ID NO: 44)
Construct I: SS2-RBD2(330-530)-GS-Linker-GS-TM1 (SEQ ID NO: 45)
Construct J: SS2-RBD2(330-530)-GS-Linker-GS-TM2 (SEQ ID NO: 46)
Construct K: SS3-RBD3(327-531)-TM2 (SEQ ID NO: 47)
Construct L: SS1-RBD2(330-521)-TM2 (SEQ ID NO: 48)
Construct M: SS1-RBD3(327-531)-C-TM3 (SEQ ID NO: 49)
Construct N: SS3-RBD3(327-531)-TM3 (SEQ ID NO: 50)
Construct O: SS3-RBD1(330-521)-TM3 (SEQ ID NO: 51)

Signal sequence
SS1: SARS-CoV-2 signal sequence (1-15):
(SEQ ID NO: 14)
MFVFLVLLPLVSSQC
SS2: hIL-2 signal sequence:
(SEQ ID NO: 15)
MYRMQLLSCIALSLALVINS
SS3: SARS-CoV-2 signal sequence (1-13):
(SEQ ID NO: 16)
MFVFLVLLPLVSS

### SARS-COV-2-RBDs

RBD1: SARS-COV-2 RBD amino acid position 330 and 521
RBD2: SARS-COV-2 RBD amino acid position 330 and 530
RBD3: SARS-COV-2 RBD amino acid position 327 and 531

RBD1:
(SEQ ID NO: 17)
PNITNLCPFGEVENATRFASVYAWNRKRISNCVADYSVLYNSASFSTFK
CYGVSPTKLNDLCFTNVYADSFVIRGDEVRQIAPGQTGKIADYNYKLPD
DFTGCVIAWNSNNLDSKVGGNYNYLYRLFRKSNLKPFERDISTEIYQAG
STPCNGVEGENCYFPLQSYGFQPTNGVGYQPYRVVVLSFELLHAP
RBD2:
(SEQ ID NO: 18)
PNITNLCPFGEVENATRFASVYAWNRKRISNCVADYSVLYNSASFSTFK
CYGVSPTKLNDLCFTNVYADSFVIRGDEVRQIAPGQTGKIADYNYKLPD
DFTGCVIAWNSNNLDSKVGGNYNYLYRLFRKSNLKPFERDISTEIYQAG
STPCNGVEGENCYFPLQSYGFQPTNGVGYQPYRVVVLSFELLHAPATVC
GPKKS
RBD3:
(SEQ ID NO: 19)
VRFPNITNLCPFGEVENATRFASVYAWNRKRISNCVADYSVLYNSASFS
TFKCYGVSPTKLNDLCFTNVYADSFVIRGDEVRQIAPGQTGKIADYNYK
LPDDFTGCVIAWNSNNLDSKVGGNYNYLYRLFRKSNLKPFERDISTEIY
QAGSTPCNGVEGENCYFPLQSYGFQPTNGVGYQPYRVVVLSFELLHAPA
TVCGPKKST
Linker
Human IgG4-CH3
(SEQ ID NO: 20)
GQPREPQVYTLPPSQEEMTKNQVSLTCLVKGFYPSDIAVEWESNGQPEN
NYKTTPPVLDSDGSFFLYSRLTVDKSRWQEGNVFSCSVMHEALHNHYTQ
KSLSLSLGK

### Transmembrane-Cytoplasm (TM-Cyt)

TM1: Human CD80* (TM-Cyt):
(SEQ ID NO: 21)
LLPSWAIT LISVNGIFVI CCLTYCFAPR CRERRRNERL
RRESVRPV
*CD80: ncbi.nlm.nih.gov/protein/NP_005182
TM2: HA(flexible-TM-Cyt):
(SEQ ID NO: 22)
GVKLESMGIYQILAIYSTVASSLVLLVSLGAISFWMCSNGSLQCRICI
TM3: COVID19 (JMD-TM):
(SEQ ID NO: 23)
GKYEQYIKWPWYIWLGFIAGLIAIVMVTIMLCCMTSCCSCLKGCCSCGS
CCKFDEDDSEPVLKGVKLHYT

The juxtamembrane domain (JMD) of spike protein is an aromatic amino acid-rich region proximal to the transmembrane domain that is highly conserved in all coronaviruses (Howard et al., JOURNAL OF VIROLOGY, March 2008, p. 2883-2894 Vol. 82, No. 6, the contents of the reference is herein incorporated by reference).

JMD:
(SEQ ID NO: 24)
GKYEQYIKWPWYIWL
TM:
(SEQ ID NO: 25)
GFIAGLIAIVMVTIMLCCMTSCCSCLKGCCSCGSCCKFDEDDSEPVLKG
VKLHYT

### Example 3

Preparation of replicon vector. Schematic construct of the alphavirus replicon is shown in FIG. 1.

Each gene of Interest DNA prepared in Example 1 and 2 was cloned into the VEEV replicon vector under the control of the subgenomic (SG) promoter. The VEEV replicon plasmid encoding each fragment was created by insertion at AscI and SbfI restriction sites to obtain the full-length VEEV TC-83 replicon construct. One example of the full length VEEV TC-83 plasmid vector is shown in FIG. 2.

Nucleotide sequences of SG promoter, 5′UTR, 3′UTR and Poly A tail are as follows. RNA sequences were obtained by using those DNA sequences as template.

SG promoter:
(SEQ ID NO: 26)
cctgaatggactacgacatagtctagtccgccaag
5′UTR:
(SEQ ID NO: 27)
ataggcggcgcatgagagaagcccagaccaattacctacccaaa
3′UTR:
(SEQ ID NO: 28)
gcgatcgcatacagcagcaattggcaagctgcttacatagaactcgcgg
cgattggcatgccgccttaaaatttttattttatttttcttttcttttc
cgaatcggattttgtttttaatatttc
Poly A tail:
(SEQ ID NO: 29)
aaaaaaaaaaaaaaaaaaaaaaaaaaaaaaaaaaaaaaaaaaaaaaaaa
aaaaaa

VEEV TC-83 Replicon nsP1-4 amino acid sequence is as follows.

(SEQ ID NO: 30)
MEKVHVDIEEDSPFLRALQRSFPQFEVEAKQVTDNDHANARAFSHLASKLIETEVDP
SDTILDIGSAPARRMYSKHKYHCICPMRCAEDPDRLYKYATKLKKNCKEITDKELDK
KMKELAAVMSDPDLETETMCLHDDESCRYEGQVAVYQDVYAVDGPTSLYHQANKG
VRVAYWIGFDTTPFMFKNLAGAYPSYSTNWADETVLTARNIGLCSSDVMERSRRGMS
ILRKKYLKPSNNVLFSVGSTIYHEKRDLLRSWHLPSVFHLRGKQNYTCRCETIVSCDG
YVVKRIAISPGLYGKPSGYAATMHREGFLCCKVTDTLNGERVSFPVCTYVPATLCDQ
MTGILATDVSADDAQKLLVGLNQRIVVNGRTQRNTNTMKNYLLPVVAQAFARWAKE
YKEDQEDERPLGLRDRQLVMGCCWAFRRHKITSIYKRPDTQTIIKVNSDFHSFVLPRI
GSNTLEIGLRTRIRKMLEEHKEPSPLITAEDIQEAKCAADEAKEVREAEELRAALPPLA
ADFEEPTLEADVDLMLQEAGAGSVETPRGLIKVTSYAGEDKIGSYAVLSPQAVLKSEK
LSCIHPLAEQVIVITHSGRKGRYAVEPYHGKVVVPEGHAIPVQDFQALSESATIVYNER
EFVNRYLHHIATHGGALNTDEEYYKTVKPSEHDGEYLYDIDRKQCVKKELVTGLGLT
GELVDPPFHEFAYESLRTRPAAPYQVPTIGVYGVPGSGKSGIIKSAVTKKDLVVSAKKE
NCAEIIRDVKKMKGLDVNARTVDSVLLNGCKHPVETLYIDEAFACHAGTLRALIAIIR
PKKAVLCGDPKQCGFFNMMCLKVHFNHEICTQVFHKSISRRCTKSVTSVVSTLFYDK
RMRTTNPKETKIVIDTTGSTKPKQDDLILTCFRGWVKQLQIDYKGNEIMTAAASQGLT
RKGVYAVRYKVNENPLYAPTSEHVNVLLTRTEDRIVWKTLAGDPWIKILTAKYPGNFT
ATIEEWQAEHDAIMRHILERPDPTDVFQNKANVCWAKALVPVLKTAGIDMTTEQWN
TVDYFETDKAHSAEIVLNQLCVRFFGLDLDSGLFSAPTVPLSIRNNHWDNSPSPNMY
GLNKEVVRQLSRRYPQLPRAVATGRVYDMNTGTLRNYDPRINLVPVNRRLPHALVLH
HNEHPQSDFSSFVSKLKGRTVLVVGEKLSVPGKKVDWLSDQPEATFRARLDLGIPGD
VPKYDIVFINVRTPYKYHHYQQCEDHAIKLSMLTKKACLHLNPGGTCVSIGYGYADR
ASESIIGAIARQFKFSRVCKPKSSHEETEVLFVFIGYDRKARTHNPYKLSSTLTNIYTGS
RLHEAGCAPSYHVVRGDIATATEGVIINAANSKGQPGGGVCGALYKKFPESFDLQPIE
VGKARLVKGAAKHIIHAVGPNFNKVSEVEGDKQLAEAYESIAKIVNDNNYKSVAIPLL
STGIFSGNKDRLTQSLNHLLTALDTTDADVAIYCRDKKWEMTLKEAVARREAVEEICIS
DDSSVTEPDAELVRVHPKSSLAGRKGYSTSDGKTFSYLEGTKFHQAAKDIAEINAMW
PVATEANEQVCMYILGESMSSIRSKCPVEESEASTPPSTLPCLCIHAMTPERVQRLKAS
RPEQITVCSSFPLPKYRITGVQKIQCSQPILFSPKVPAYIHPRKYLVETPPVEETPESPAE
NQSTEGTPEQPALVNVDATRTRMPEPIIIEEEEEDSISLLSDGPTHQVLQVEADIHGSPS
VSSSSWSIPHASDFDVDSLSILDTLDGASVTSGAVSAETNSYFARSMEFRARPVPAPRT
VFRNPPHPAPRTRTPPLAHSRASSRTSLVSTPPGVNRVITREELEALTPSRAPSRSASRTS
LVSNPPGVNRVITREEFEAFVAQQQXRFDAGAYIFSSDTGQGHLQQKSVRQTVLSEVV
LERTELEISYAPRLDQEKEELLRKKLQLNPTPANRSRYQSRRVENMKAITARRILQGLG
HYLKAEGKVECYRTLHPVPLYSSSVNRAFSSPKVAVEACNAMLKENFPTVASYCIIPE
YDAYLDMVDGASCCLDTASFCPAKLRSFPKKHSYLEPTIRSAVPSAIQNTLQNVLAAA
TKRNCNVTQMRELPVLDSAAFNVECFKKYACNNEYWETFKENPIRLTEENVVNYITK
LKGPKAAALFAKTHNLNMLQDIPMDRFVMDLKRDVKVTPGTKHTEERPKVQVIQA
ADPLATADLCGIHRELVRRLNAVLLPNIHTLFDMSAEDFDAIIAEHFQPGDCVLETDIA
SFDKSEDDAMALTALMILEDLGVDAELLTLIEAAFGEISSIHLPTKTKFKFGAMMKSG
MFLTLFVNTVINIVIASRVLRERLTGSPCAAFIGDDNIVKGVKSDKLMADRCATWLNM
EVKIIDAVVGEKAPYFCGGFILCDSVTGTACRVADPLKRLFKLGKPLAVDDEHDDDRR
RALHEESTRWNRVGILPELCKAVESRYETVGTSIIVMAMTTLASSVKSFSYLRGAPITL
YG

Amino acid sequence corresponding to nsP3 is underlined.

In this example, amino acid sequence of nsP3 which is corresponding from 1330-1886 in SEQ ID NO: 30 was replaced with the sequence shown below. The underlined sequence was different from SEQ ID NO: 30.

(SEQ ID NO: 31)
APSYHVVRGDIATATEGVIINAANSKGQPGGGVCGALYKKFPESFDLQPIEVGKARLV
KGAAKHIIHAVGPNFNKVSEVEGDKQLAEAYESIAKIVNDNNYKSVAIPLLSTGIFSGN
KDRLTQSLNHLLTALDTTDADVAIYCRDKKWEMTLKEAVARREAVEEICISDDSSVTE
PDAELVRVHPKSSLAGRKGYSTSDGKTFSYLEGTKFHQAAKDIAEINAMWPVATEAN
EQVCMYILGKSMSSIRSKCPVEESEASTPPSTLPCLCIHAMTPERVQRLKASRPEQITV
CSSFPLPKYRITGVQKIQCSQPILFSPKVPAYIHPRKYLVETPPVDETPEPSAENQSTEGT
PEQPPLITEDETRTRTPEPIIIEEEEEDSISLLSDGPTHQVLQVEADIHGPPSVSSSSWSIP
HASDFDVDSLSILDTLEGASVTSGATSAETNSYFAKSMEFLARPVPAPRTVFRNPPHPA
PRTRTPSLAPSRACSRTSLVSTPPGVNRVITREELEALTPSRTPSRSVSRTSLVSNPPGVN
RVITREEFEAFVAQQQXRFDAGA

### Example 4

### Preparation of Alphavirus Replicon Particles

10 μg of the full-length replicon plasmid prepared in Example 3, 1 μg of VEEV Env expression plasmid and 1 μg of VEEV Capsid NLS mutant (or 1μg VEEV Capsid expression plasmid) was transfected into HEK293T cells. The supernatant was harvested 48-96 hours after transfection. The replicon particle was purified by using an ion exchange column. HET293T or Vero cells were infected with dilutions of the purified particle preparation to determine the infectious titer. The purified replicon particles are used for producing antigens for diagnosis and vaccination.

### Example 5

### Expression of RBD in HEK293T Cells

293T cells (6.25×10\hat{ }5 cells/well) were transfected with Fectopro transfection reagent (Polyplus) and 2 μg of each Replicon plasmid. Western blotting was performed on the supernatant directly without purification of particles (left) or on cell lysate fractions (right) 72 h after transfection using antisera reactive with SARS-COV-2-RBD as a primary antibody and goat anti-rabbit immunoglobulins linked to horseradish peroxidase as a secondary antibody. Also Western blotting was performed for confirming the expression of antigen from Construct K and Construct O in the cell lysate fractions 72 h after transfection using antisera reactive with SARS-COV-2-RBD as a primary antibody and goat anti-rabbit immunoglobulins linked to horseradish peroxidase as a secondary antibody.

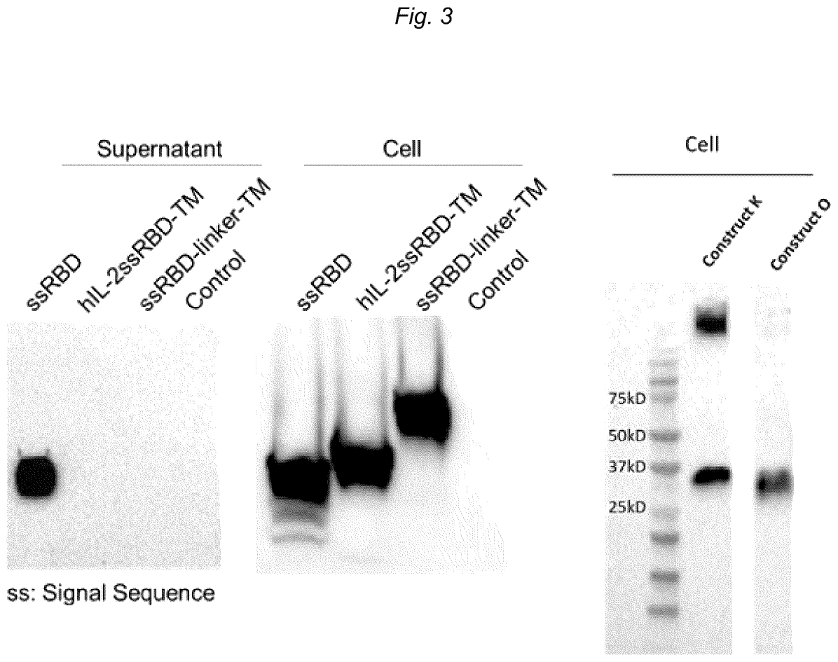

The results are shown in FIG. 3.

The abbreviation means as follows:
ssRBD: construct A
hIL-2ssRBD-TM: construct 1 (mouse HA)
ssRBD-linker-TM: construct C (TM1: mouse CD80, Linker: mouse IgG4-CH3)

Mouse: CD80(TM-Cyt)
(SEQ ID NO: 32)
TLVLFGAGFGAVITVVVIVVIIKCFCKHRSCFRRNEASRETNNSLTFGP
EEALAEQTVFL
Mouse IgG4-CH3
(SEQ ID NO: 33)
GRPKAPQVYTIPPPKEQMAKDKVSLTCMITNFFPEDITVEWQWNGQPAE
NYKNTQPIMDTDGSYFVYSKLNVQKSNWEAGNTFTCSVLHEGLHNHHTE
KSLSHSPGK

Expressions were confirmed for all tested particles in the cells. As expected the ssRBD without a transmembrane sequence was secreted into the medium of the cell culture, whereas each of the RBD constructs linked to a transmembrane sequence was retained in the cells and not secreted into the media.

### Example 6

### Immunogenicity

The following Replicon Particle Vaccines prepared in Example 4 were used.
VRep ssRBD: VEEV particle packaging construct A
VRep sshIL2 RBD HA: VEEV particle packaging construct 1
VRep ssRBD mIgG4 CD80: VEEV Replicon particle packaging Construct C (TM1: mouse CD80, Linker: mouse IgG4-CH3)
Control: VEEV Replicon without gene of interest
DNA vaccine: DNA expressing the spike protein of SARS-COV-2

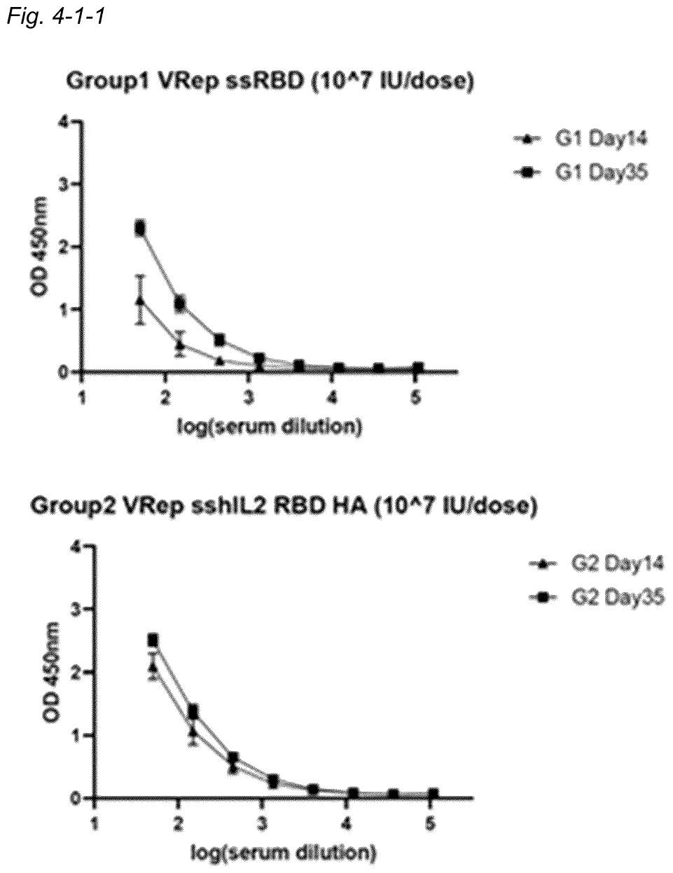

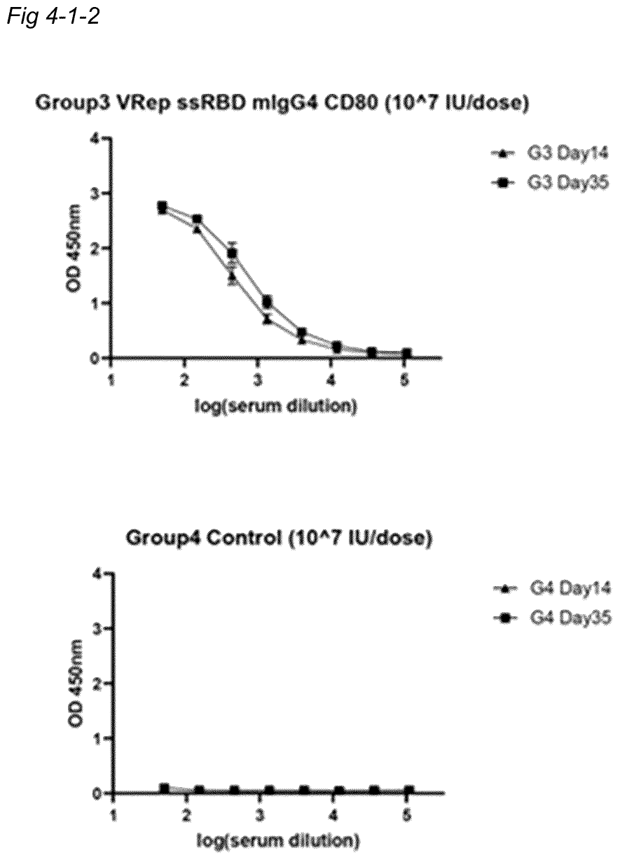

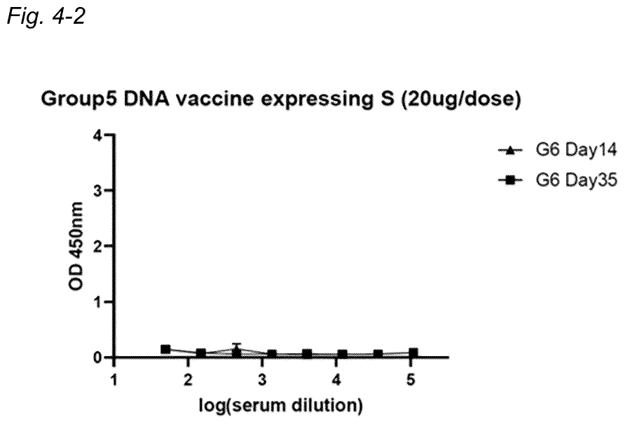

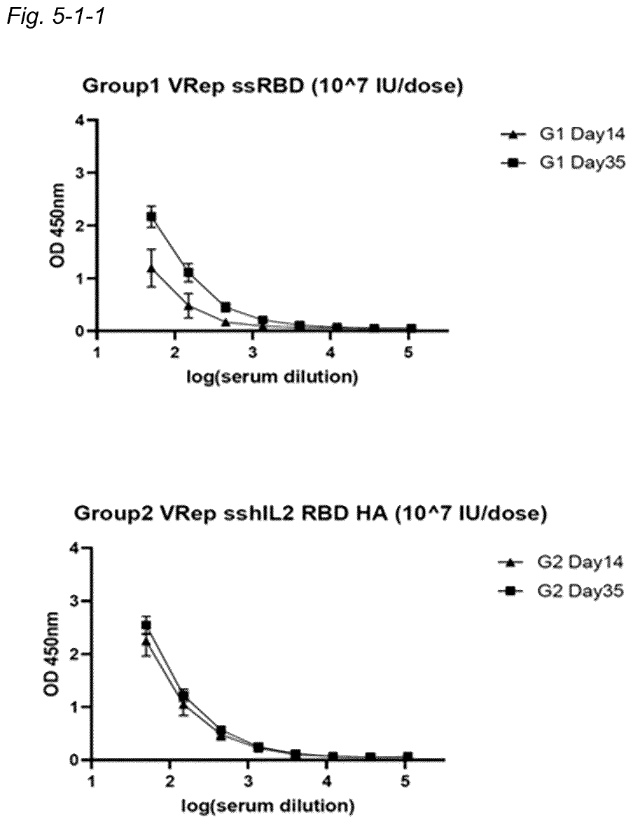

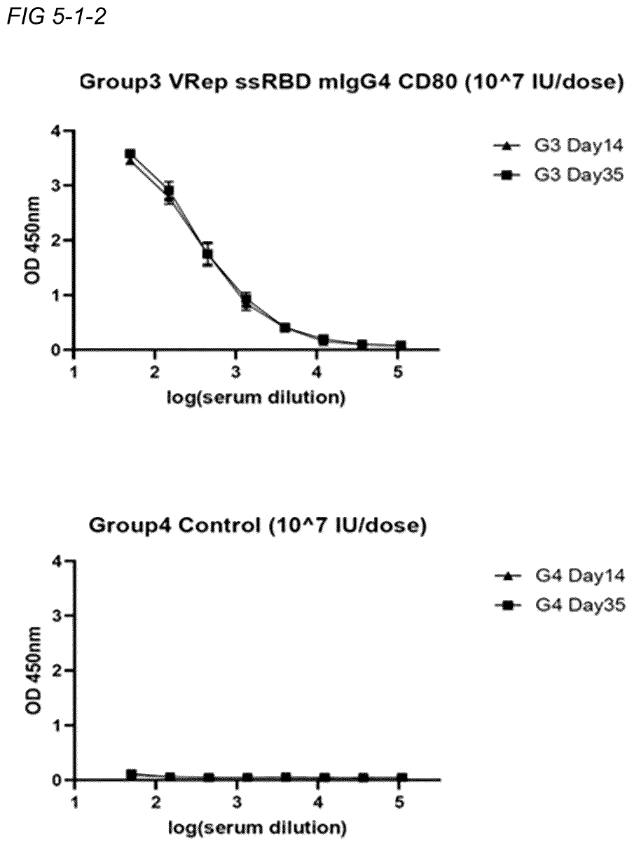

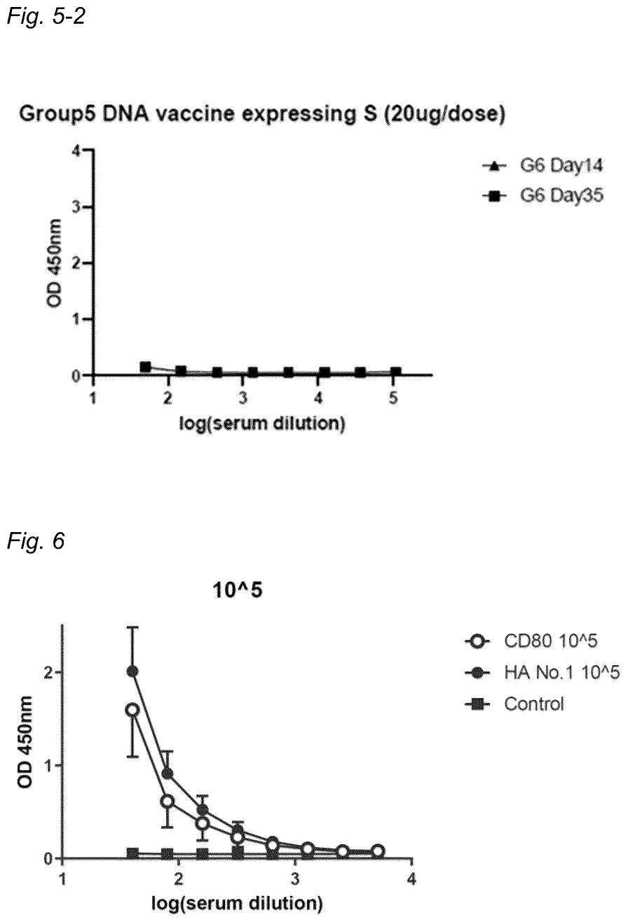

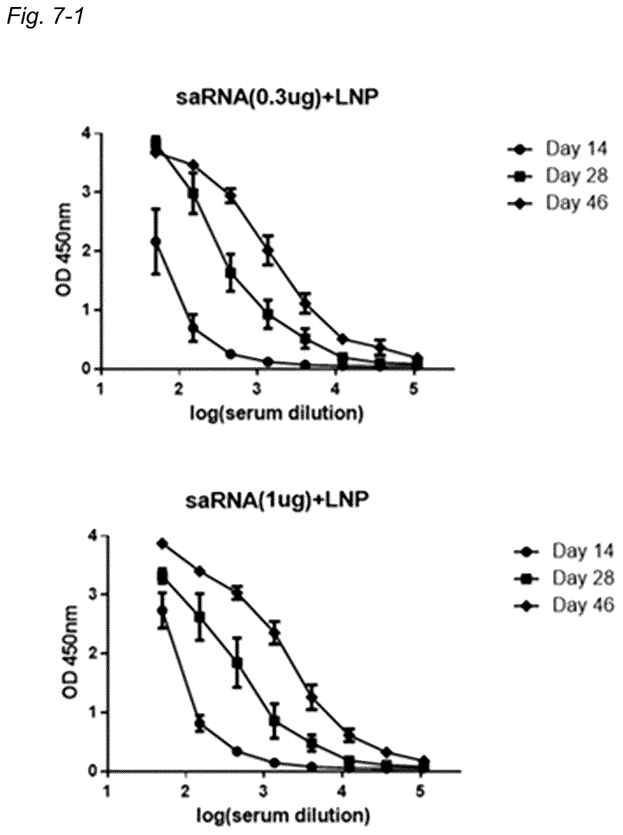

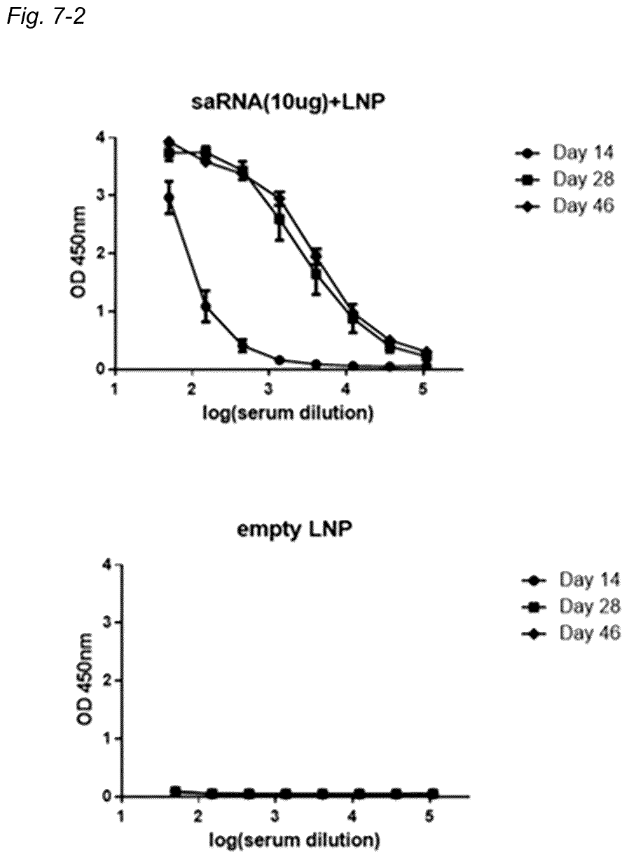

Mice were immunized intramuscularly three times (on Day 0, Day 7 and Day 14) with the indicated Replicon particles (RBD or RBD-linker-TM, 10\hat{ }7 IU/dose), a control Replicon particle or a DNA vaccine (20 μg/dose), and sera were collected on Dayl4 (7 days after 2nd immunization) and Day 35 (21 days after 3nd immunization). These sera were examined in ELISA assays performed against a SARS-COV-2-Spike S1 or RBD antigen. The symbols show the average of the five mice in each group, and error bars show the s.e.m. The curve fit was calculated by Prism software. These results are shown in FIGS. 4-1-1, 4-1-2 and 4-2 and FIGS. 5-1-1, 5-1-2 and 5-2.

FIGS. 4-1-1, 4-1-2 and 4-2 and FIGS. 5-1-1, 5-1-2 and 5-2 show that significantly higher titers of antibody against recombinant Coronavirus RBD and Spike S1 were achieved after immunization with replicon vaccines. In contrast, very low titers of antibody were induced with the DNA vaccine.

### Example 7

### Immunogenicity

The following Replicon Particle Vaccines prepared in Example 4 were used.
CD80: VEEV particle packaging Construct C
HA No.1: VEEV particle packaging Construct K
Control: PBS

Mice were immunized intramuscularly with the indicated Replicon vaccines (CD80 or HA No.1, 10\hat{ }5 IU/dose) or PBS, and sera were collected 7 days after immunization. These sera were subject to ELISA analysis performed against a SARS-COV-2-Spike S1 antigen. The symbols show the mean of the five mice in each group, and error bars show the s.e.m. These results are shown in FIG. 6.

FIG. 6 shows that significantly higher titers of antibody against recombinant Coronavirus Spike S1 were achieved after immunization with replicon vaccines.

### Example 8

Preparation of Self-Amplifying RNA (saRNA) Encapsulated in Lipid Nanoparticles (LNP)

The vector comprising the DNA sequence encoding construct C prepared in Example 3 was used. The DNA was linearized and used as the template. T7 in vitro transcription was conducted based on protocols provided by the T7 transcription kit (RiboMax™ Express Large Scale RNA production System, Promega, (WI USA)). The linear DNA template was mixed with T7 enzyme and rNTPs to synthesize RNA. The purified RNA product was capped using vaccinia capping enzyme to give self-amplifying RNA.

The obtained saRNA was encapsulated in lipid nanoparticles to give saRNA (RBD-CD80TM) particles. Lipid nanoparticles with no RNA was used as control.

### Example 9

### Immunization

saRNAs formulated with LNP prepared in Example 8 were used.

### Amount of Immunization

0.3 μg saRNA (RBD-CD80TM)
1 μg saRNA (RBD-CD80TM)
10 μg saRNA (RBD-CD80TM)
no RNA (control)

The indicated amount of saRNA or no RNA (control) formulated with LNP (Genvoy) were used. The 4-6 weeks old mice (n=5 per group) were immunized with saRNA formulated with LNP on Day 0 and Day 28 by intramuscularly injections.

The mice were bleed on Day 14, 28 and 46. The antibody titer against S1 protein in the sera from the immunized mice were measured by ELISA. The plate were coated with S1 protein (Sinobio 100 ng/ml). Results are shown in FIG. 6.

### Example 10

The SARS-COV-2 RBD may be derived from SARS-COV-2 mutants. The following amino acid sequences of constructions comprising the SARS-COV-2 signal sequence, SARS-COV-2 RBD derived from a SARS-COV-2 mutant, and human HA (flexible-TM-Cyt) are also used for generating alphavirus replicon as shown in FIG. 1. In this example the gene of interest is replaced with the polynucleotide encoding the following amino acid sequence. Self-amplifying RNA (saRNA) encapsulated in lipid nanoparticles (LNP) is generated in the same manner as Example 8.

Construct derived from SARS-CoV-2 E484K_N501Y_K417T mutant
(Brazil Strain Mutant)
(SEQ ID NO: 34)
MFVFLVLLPLVSSVRFPNITNLCPFGEVENATRFASVYAWNRKRISNCVADYSVLYN
SASFSTFKCYGVSPTKLNDLCFTNVYADSFVIRGDEVRQIAPGQTGTIADYNYKLPDD
FTGCVIAWNSNNLDSKVGGNYNYLYRLFRKSNLKPFERDISTEIYQAGSTPCNGVKG
FNCYFPLQSYGFQPTYGVGYQPYRVVVLSFELLHAPATVCGPKKSTGVKLESMGIYQ
ILAIYSTVASSLVLLVSLGAISFWMCSNGSLQCRICI
Construct derived from SARS-CoV-2 E484K_N501Y_K417N mutant
(South African Mutant)
(SEQ ID NO: 35)
MFVFLVLLPLVSSVRFPNITNLCPFGEVENATRFASVYAWNRKRISNCVADYSVLYN
SASFSTFKCYGVSPTKLNDLCFTNVYADSFVIRGDEVRQIAPGQTGNIADYNYKLPD
DFTGCVIAWNSNNLDSKVGGNYNYLYRLFRKSNLKPFERDISTEIYQAGSTPCNGVK
GFNCYFPLQSYGFQPTYGVGYQPYRVVVLSFELLHAPATVCGPKKSTGVKLESMGIY
QILAIYSTVASSLVLLVSLGAISFWMCSNGSLQCRICI
Construct derived from SARS-CoV-2 E484K mutant
(SEQ ID NO 36)
MFVFLVLLPLVSSVRFPNITNLCPFGEVFNATRFASVYAWNRKRISNCVADYSVLYN
SASFSTFKCYGVSPTKLNDLCFTNVYADSFVIRGDEVRQIAPGQTGKIADYNYKLPD
DFTGCVIAWNSNNLDSKVGGNYNYLYRLFRKSNLKPFERDISTEIYQAGSTPCNGVK
GFNCYFPLQSYGFQPTNGVGYQPYRVVVLSFELLHAPATVCGPKKSTGVKLESMGIY
QILAIYSTVASSLVLLVSLGAISFWMCSNGSLQCRICI

Specific Embodiments provided by this disclosure are as follows:
(1) An isolated polynucleotide, which encodes alphavirus non-structural proteins nsP1, nsP2, nsP3 and nsP4 and a polypeptide comprising a coronavirus protein fused to a signal sequence and/or transmembrane domain.
(2) The polynucleotide of (1), wherein the coronavirus protein is a spike (S) protein, nucleocapsid (N) protein, membrane (M) protein, small envelope (E) protein or a combination thereof.
(3) The polynucleotide of (1), wherein the coronavirus protein is a spike (S) protein.
(4) The polynucleotide of (1), wherein the coronavirus protein is a S1 and/or S2 subunit of a spike (S) protein.
(5) The polynucleotide of (4), wherein the coronavirus protein is a S1 subunit in a spike (S) protein.
(6) The polynucleotide of (5), wherein the coronavirus protein is a receptor binding domain of the S1 subunit.
(7) The polynucleotide of any one of (1) to (6), wherein the transmembrane domain is derived from Influenza Hemagglutinin (HA), CD80 or TLR4.
(8) The polynucleotide any one of (1) to (6), wherein the transmembrane domain is a modified transmembrane domain derived from coronavirus structural protein.
(9) The polynucleotide of (8), the modified transmembrane domain comprises juxtamembrane domain and transmembrane domain of SARS-COV-2 Spike (S).
(10) The polynucleotide of any one of (1) to (9), wherein coronavirus protein is fused to a signal sequence and transmembrane domain.
(11) The polynucleotide of any one of (1) to (10), wherein the signal sequence is derived from human IL-2.
(12) The polynucleotide of any one of (1) to (10), wherein the signal sequence is derived from SARS-COV-2 spike protein.
(13) The polynucleotide of any one of (1) to (12), wherein the transmembrane domain and/or signal sequence is fused to the coronavirus protein by a linker.
(14) The polynucleotide of (13) wherein the linker is IgG4CH3 and/or short linker.
(15) The polynucleotide of any one of (1) to (14), wherein the coronavirus is SARS-COV-2.
(16) The polynucleotide of (15), wherein the polypeptide encodes an amino acid sequence selected from SEQ ID NOs 1, 5, 6, 8, 9, 11, 12, 13, 34, 35 and 36.
(17) The polynucleotide of (15), wherein the polypeptide encodes an amino acid sequence selected from SEQ ID NOs 37-51.
(18) The polynucleotide of any one of (1) to (17), wherein the polynucleotide is RNA.
(19) The polynucleotide of any one of (1) to (17), wherein the polynucleotide is DNA.
(20) A vector comprising the polynucleotide of any one of (1) to (19).
(21) The vector of (20), which comprises a promoter, 5′ UTR, polynucleotide encoding alphavirus non-structural proteins nsP1, nsP2, nsP3 and nsP4, SG promoter, a gene of interest encoding the polypeptide comprising a coronavirus protein fused to a signal sequence and/or transmembrane domain, 3′UTR and poly A tail.
(22) A vaccine composition comprising the polynucleotide or vector of any one of (1) to (21) and a pharmaceutically acceptable carrier.
(23) The vaccine composition of (22), wherein the pharmaceutically acceptable carrier is a delivery vehicle.
(24) The vaccine composition of (23), wherein the delivery vehicle is a particle consisting of one or more alphavirus structural proteins or a lipid delivery system.
(25) Use of the polynucleotide or vector of any one of (1) to (21) for the manufacture of a medicament.
(26) Use of (25), wherein the medicament is for inducing immunomodulation in a subject.

(27) Use of (25), wherein the medicament is for treating or preventing a subject from conditions caused by coronavirus infection.
(28) A method of immunomodulation in a subject, comprising administering an immunologically effective amount of the vaccine composition of any one of (22) to (24), to the subject in need thereof.
(29) A method of treating, preventing and/or immunizing against coronavirus viral infection in a subject, comprising administering an effective amount of the vaccine composition of any one of (22) to (24), to the subject in need thereof.
(30) A polypeptide comprising a coronavirus structural protein fused to a signal sequence and/or heterologous transmembrane domain.

What is claimed is:

1. An isolated polynucleotide which encodes a polypeptide comprising a receptor binding domain (rbd) of an S1 subunit in a spike protein of a sars-cov-2 fused to a transmembrane domain and optionally to a signal sequence, wherein the transmembrane domain is heterologous to the sars-cov-2.

2. The polynucleotide of claim 1, wherein the coronavirus protein is a spike (S) protein.

3. The polynucleotide of claim 1, wherein the coronavirus protein is a S1 and/or S2 subunit of a spike (S) protein.

4. The polynucleotide of claim 3, wherein the coronavirus protein is a S1 subunit in a spike (S) protein.

5. The polynucleotide of claim 4, wherein the coronavirus protein is a receptor binding domain of the S1 subunit.

6. The polynucleotide of claim 5, wherein the transmembrane domain is derived from Influenza Hemagglutinin (HA), CD80, or a modified transmembrane domain derived from coronavirus structural protein.

7. The polynucleotide of claim 6, wherein the transmembrane domain is derived from Influenza Hemagglutinin (HA).

8. The polynucleotide of claim 6, the modified transmembrane domain comprises juxtamembrane domain and transmembrane domain of COVID-19 Spike (S).

9. The polynucleotide of claim 1, wherein coronavirus protein is fused to a signal sequence and transmembrane domain.

10. The polynucleotide of claim 1, wherein the signal sequence is derived from human IL-2 or COVID-19 spike protein.

11. The polynucleotide of claim 1, wherein the transmembrane domain and/or signal sequence is fused to the coronavirus protein by a linker.

12. The polynucleotide of claim 11, wherein the linker is IgG4CH3 and/or short linker.

13. The polynucleotide of claim 1, wherein the coronavirus is COVID-19.

14. The polynucleotide of claim 1, wherein the polynucleotide is RNA.

15. A vector comprising the polynucleotide of claim 1.

16. The vector of claim 15, which comprises a promoter, 5′ UTR, polynucleotide encoding alphavirus non-structural proteins nsp1, nsp2, nsp3 and nsp4, SG promoter, a gene of interest encoding the polypeptide comprising a coronavirus protein fused to a signal sequence and/or transmembrane domain, 3′UTR and poly A tail.

17. A vaccine composition comprising the polynucleotide or vector of claim 1 and a pharmaceutically acceptable delivery vehicle.

18. The vaccine composition of claim 17, wherein the delivery vehicle is a particle consisting of one or more alphavirus structural proteins or a lipid delivery system.

19. A method of treating, preventing and/or immunizing against coronavirus viral infection in a subject, comprising administering an effective amount of the vaccine of claim 17 to the subject in need thereof.

20. The polynucleotide according to claim 1, wherein the polypeptide further comprises alphavirus non-structural proteins nsp1, nsp2, nsp3 and nsp4, wherein the alphavirus is Venezuelan Equine Encephalitis Virus or Chikungunya virus.


SEQUENCE LISTING (51 sequences)

SEQ ID NO: 1 - protein, 272 aa, synthetic construct
  REGION 1..272: note = Fused protein comprising hIL-2 signal sequence, COVID-19-RBD and HA
MYRMQLLSCIALSLALVTNSVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSGVKLESMGIYQILAIYSTVASSLVLLVSLGAISFWMCSNGSLQCRICI

SEQ ID NO: 2 - protein, 20 aa, synthetic construct
  REGION 1..20: note = Human IL-2 signal sequence
MYRMQLLSCIALSLALVTNS

SEQ ID NO: 3 - protein, 204 aa, synthetic construct
  REGION 1..204: note = COVID19-RBD
VRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKS

SEQ ID NO: 4 - protein, 48 aa, synthetic construct
  REGION 1..48: note = Inflenza HA (flexible-TM-Cyt)
GVKLESMGIYQILAIYSTVASSLVLLVSLGAISFWMCSNGSLQCRICI

SEQ ID NO: 5 - protein, 274 aa, synthetic construct
  REGION 1..274: note = Fused protein comprising hIL-2 signal sequence, COVID-19-RBD and HA(flexible-TM-Cyt).
MYRMQLLSCIALSLALVTNSCVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSCGVKLESMGIYQILAIYSTVASSLVLLVSLGAISFWMCSNGSLQCRICI

SEQ ID NO: 6 - protein, 383 aa, synthetic construct
  REGION 1..383: note = Fused protein comprising hIL-2 signal sequence, COVID-19-RBD, human IgG4CH3 and HA(flexible-TM-Cyt)
MYRMQLLSCIALSLALVTNSVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSGSGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGSGVKLESMGIYQILAIYSTVASSLVLLVSLGAISFWMCSNGSLQCRICI

SEQ ID NO: 7 - protein, 107 aa, synthetic construct
  REGION 1..107: note = Human IgG4 CH3 (linker)
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 385 aa, synthetic construct
  REGION 1..385: note = Fused protein comprising hIL-2 signal sequence, COVID-19-RBD, human IgG4CH3 and HA (flexible-TM-Cyt)
MYRMQLLSCIALSLALVTNSCVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSCGSGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGSGVKLESMGIYQILAIYSTVASSLVLLVSLGAISFWMCSNGSLQCRICI

SEQ ID NO: 9 - protein, 435 aa, synthetic construct
  REGION 1..435: note = Fused protein comprising hIL-2 signal sequence, COVID-19-RBD, and human TLR4(TM-TIR)
MYRMQLLSCIALSLALVTNSVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSGSKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 10 - protein, 209 aa, synthetic construct
  REGION 1..209: note = Human TLR4 (TM-Toll/interleukin-1 receptor domain)
KTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 11 - protein, 437 aa, synthetic construct
  REGION 1..437: note = Fused protein comprising hIL-2 signal sequence, COVID-19-RBD and human TLR4 (TM-TIR)
MYRMQLLSCIALSLALVTNSCVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSCGSKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 12 - protein, 544 aa, synthetic construct
  REGION 1..544: note = Fused protein comprising hIL-2 signal sequence, COVID-19-RBD, human IgG4CH3, and Human TLR4(TM-TIR)
MYRMQLLSCIALSLALVTNSVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSGSGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGSKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 13 - protein, 546 aa, synthetic construct
  REGION 1..546: note = Fused protein comprising hIL-2 signal sequence, COVID19-RBD, human IgG4CH3, and Human TLR4(TM-TIR)
MYRMQLLSCIALSLALVTNSCVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSCGSGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGSKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 14 - protein, 15 aa, synthetic construct
  REGION 1..15: note = COVID-19 signal sequence (1-15)
MFVFLVLLPLVSSQC

SEQ ID NO: 15 - protein, 20 aa, synthetic construct
  REGION 1..20: note = Human IL-2 signal sequence
MYRMQLLSCIALSLALVTNS

SEQ ID NO: 16 - protein, 13 aa, synthetic construct
  REGION 1..13: note = COVID-19 signal sequence (1-13)
MFVFLVLLPLVSS

SEQ ID NO: 17 - protein, 192 aa, synthetic construct
  REGION 1..192: note = COVID-19 RBD (AA 330-521)
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAP

SEQ ID NO: 18 - protein, 201 aa, synthetic construct
  REGION 1..201: note = COVID-19 RBD (AA 330-530)
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKS

SEQ ID NO: 19 - protein, 205 aa, synthetic construct
  REGION 1..205: note = COVID-19 RBD (AA 327-531)
VRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKST

SEQ ID NO: 20 - protein, 107 aa, synthetic construct; identical to SEQ ID NO: 7
  REGION 1..107: note = Human IgG4-CH3 (Linker)

SEQ ID NO: 21 - protein, 46 aa, synthetic construct
  REGION 1..46: note = Human CD80 transmembrane-cytoplasm
LLPSWAITLISVNGIFVICCLTYCFAPRCRERRRNERLRRESVRPV

SEQ ID NO: 22 - protein, 48 aa, synthetic construct; identical to SEQ ID NO: 4
  REGION 1..48: note = Influenza HA (flexible-TM-Cyt)

SEQ ID NO: 23 - protein, 70 aa, synthetic construct
  source 1..70: note = COVID-19 juxtamembrane domain and transmembrane domain
GKYEQYIKWPWYIWLGFIAGLIAIVMVTIMLCCMTSCCSCLKGCCSCGSCCKFDEDDSEPVLKGVKLHYT

SEQ ID NO: 24 - protein, 15 aa, synthetic construct
  REGION 1..15: note = Juxtamembrane domain of COVID-19 spike protein
GKYEQYIKWPWYIWL

SEQ ID NO: 25 - protein, 55 aa, synthetic construct
  REGION 1..55: note = Transmembrane domain of COVID-19 spike protein
GFIAGLIAIVMVTIMLCCMTSCCSCLKGCCSCGSCCKFDEDDSEPVLKGVKLHYT

SEQ ID NO: 26 - DNA, 35 bp, synthetic construct
  misc_feature 1..35: note = SG promoter
  source 1..35: mol_type = other DNA
cctgaatggactacgacatagtctagtccgccaag

SEQ ID NO: 27 - DNA, 44 bp, synthetic construct
  misc_feature 1..44: note = 5' UTR
  source 1..44: mol_type = other DNA
ataggcggcgcatgagagaagcccagaccaattacctacccaaa

SEQ ID NO: 28 - DNA, 125 bp, synthetic construct
  misc_feature 1..125: note = 3' UTR
  source 1..125: mol_type = other DNA
gcgatcgcatacagcagcaattggcaagctgcttacatagaactcgcggcgattggcatgccgccttaaaatttttattttatttttcttttcttttccgaatcggattttgtttttaatatttc

SEQ ID NO: 29 - DNA, 55 bp, synthetic construct
  misc_feature 1..55: note = Poly A tail
  source 1..55: mol_type = other DNA
aaaaaaaaaaaaaaaaaaaaaaaaaaaaaaaaaaaaaaaaaaaaaaaaaaaaaaa

SEQ ID NO: 30 - protein, 2493 aa, synthetic construct
  REGION 1..2493: note = VEEV TC-83 Replicon nsP1-4 amino acid sequence
  SITE 1880: note = misc_feature - Xaa can be any naturally occurring amino acid
MEKVHVDIEEDSPFLRALQRSFPQFEVEAKQVTDNDHANARAFSHLASKLIETEVDPSDTILDIGSAPARRMYSKHKYHCICPMRCAEDPDRLYKYATKLKKNCKEITDKELDKKMKELAAVMSDPDLETETMCLHDDESCRYEGQVAVYQDVYAVDGPTSLYHQANKGVRVAYWIGFDTTPFMFKNLAGAYPSYSTNWADETVLTARNIGLCSSDVMERSRRGMSILRKKYLKPSNNVLFSVGSTIYHEKRDLLRSWHLPSVFHLRGKQNYTCRCETIVSCDGYVVKRIAISPGLYGKPSGYAATMHREGFLCCKVTDTLNGERVSFPVCTYVPATLCDQMTGILATDVSADDAQKLLVGLNQRIVVNGRTQRNTNTMKNYLLPVVAQAFARWAKEYKEDQEDERPLGLRDRQLVMGCCWAFRRHKITSIYKRPDTQTIIKVNSDFHSFVLPRIGSNTLEIGLRTRIRKMLEEHKEPSPLITAEDIQEAKCAADEAKEVREAEELRAALPPLAADFEEPTLEADVDLMLQEAGAGSVETPRGLIKVTSYAGEDKIGSYAVLSPQAVLKSEKLSCIHPLAEQVIVITHSGRKGRYAVEPYHGKVVVPEGHAIPVQDFQALSESATIVYNEREFVNRYLHHIATHGGALNTDEEYYKTVKPSEHDGEYLYDIDRKQCVKKELVTGLGLTGELVDPPFHEFAYESLRTRPAAPYQVPTIGVYGVPGSGKSGIIKSAVTKKDLVVSAKKENCAEIIRDVKKMKGLDVNARTVDSVLLNGCKHPVETLYIDEAFACHAGTLRALIAIIRPKKAVLCGDPKQCGFFNMMCLKVHFNHEICTQVFHKSISRRCTKSVTSVVSTLFYDKRMRTTNPKETKIVIDTTGSTKPKQDDLILTCFRGWVKQLQIDYKGNEIMTAAASQGLTRKGVYAVRYKVNENPLYAPTSEHVNVLLTRTEDRIVWKTLAGDPWIKILTAKYPGNFTATIEEWQAEHDAIMRHILERPDPTDVFQNKANVCWAKALVPVLKTAGIDMTTEQWNTVDYFETDKAHSAEIVLNQLCVRFFGLDLDSGLFSAPTVPLSIRNNHWDNSPSPNMYGLNKEVVRQLSRRYPQLPRAVATGRVYDMNTGTLRNYDPRINLVPVNRRLPHALVLHHNEHPQSDFSSFVSKLKGRTVLVVGEKLSVPGKKVDWLSDQPEATFRARLDLGIPGDVPKYDIVFINVRTPYKYHHYQQCEDHAIKLSMLTKKACLHLNPGGTCVSIGYGYADRASESIIGAIARQFKFSRVCKPKSSHEETEVLFVFIGYDRKARTHNPYKLSSTLTNIYTGSRLHEAGCAPSYHVVRGDIATATEGVIINAANSKGQPGGGVCGALYKKFPESFDLQPIEVGKARLVKGAAKHIIHAVGPNFNKVSEVEGDKQLAEAYESIAKIVNDNNYKSVAIPLLSTGIFSGNKDRLTQSLNHLLTALDTTDADVAIYCRDKKWEMTLKEAVARREAVEEICISDDSSVTEPDAELVRVHPKSSLAGRKGYSTSDGKTFSYLEGTKFHQAAKDIAEINAMWPVATEANEQVCMYILGESMSSIRSKCPVEESEASTPPSTLPCLCIHAMTPERVQRLKASRPEQITVCSSFPLPKYRITGVQKIQCSQPILFSPKVPAYIHPRKYLVETPPVEETPESPAENQSTEGTPEQPALVNVDATRTRMPEPIIIEEEEEDSISLLSDGPTHQVLQVEADIHGSPSVSSSSWSIPHASDFDVDSLSILDTLDGASVTSGAVSAETNSYFARSMEFRARPVPAPRTVFRNPPHPAPRTRTPPLAHSRASSRTSLVSTPPGVNRVITREELEALTPSRAPSRSASRTSLVSNPPGVNRVITREEFEAFVAQQQXRFDAGAYIFSSDTGQGHLQQKSVRQTVLSEVVLERTELEISYAPRLDQEKEELLRKKLQLNPTPANRSRYQSRRVENMKAITARRILQGLGHYLKAEGKVECYRTLHPVPLYSSSVNRAFSSPKVAVEACNAMLKENFPTVASYCIIPEYDAYLDMVDGASCCLDTASFCPAKLRSFPKKHSYLEPTIRSAVPSAIQNTLQNVLAAATKRNCNVTQMRELPVLDSAAFNVECFKKYACNNEYWETFKENPIRLTEENVVNYITKLKGPKAAALFAKTHNLNMLQDIPMDRFVMDLKRDVKVTPGTKHTEERPKVQVIQAADPLATADLCGIHRELVRRLNAVLLPNIHTLFDMSAEDFDAIIAEHFQPGDCVLETDIASFDKSEDDAMALTALMILEDLGVDAELLTLIEAAFGEISSIHLPTKTKFKFGAMMKSGMFLTLFVNTVINIVIASRVLRERLTGSPCAAFIGDDNIVKGVKSDKLMADRCATWLNMEVKIIDAVVGEKAPYFCGGFILCDSVTGTACRVADPLKRLFKLGKPLAVDDEHDDDRRRALHEESTRWNRVGILPELCKAVESRYETVGTSIIVMAMTTLASSVKSFSYLRGAPITLYG

SEQ ID NO: 31 - protein, 557 aa, synthetic construct
  REGION 1..557: note = VEEV TC-83 nsp3 E242K
  SITE 551: note = misc_feature - Xaa can be any naturally occurring amino acid
APSYHVVRGDIATATEGVIINAANSKGQPGGGVCGALYKKFPESFDLQPIEVGKARLVKGAAKHIIHAVGPNFNKVSEVEGDKQLAEAYESIAKIVNDNNYKSVAIPLLSTGIFSGNKDRLTQSLNHLLTALDTTDADVAIYCRDKKWEMTLKEAVARREAVEEICISDDSSVTEPDAELVRVHPKSSLAGRKGYSTSDGKTFSYLEGTKFHQAAKDIAEINAMWPVATEANEQVCMYILGKSMSSIRSKCPVEESEASTPPSTLPCLCIHAMTPERVQRLKASRPEQITVCSSFPLPKYRITGVQKIQCSQPILFSPKVPAYIHPRKYLVETPPVDETPEPSAENQSTEGTPEQPPLITEDETRTRTPEPIIIEEEEEDSISLLSDGPTHQVLQVEADIHGPPSVSSSSWSIPHASDFDVDSLSILDTLEGASVTSGATSAETNSYFAKSMEFLARPVPAPRTVFRNPPHPAPRTRTPSLAPSRACSRTSLVSTPPGVNRVITREELEALTPSRTPSRSVSRTSLVSNPPGVNRVITREEFEAFVAQQQXRFDAGA

SEQ ID NO: 32 - protein, 60 aa, synthetic construct
  REGION 1..60: note = Mouse CD80 (TM-Cyt)
TLVLFGAGFGAVITVVVIVVIIKCFCKHRSCFRRNEASRETNNSLTFGPEEALAEQTVFL

SEQ ID NO: 33 - protein, 107 aa, synthetic construct
  REGION 1..107: note = Mouse IgG4-CH3
GRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 34 - protein, 266 aa, synthetic construct
  REGION 1..266: note = Construct derived from COVID-19 E484K_N501Y_K417T mutant (Brazil Strain Mutant)
MFVFLVLLPLVSSVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTGVKLESMGIYQILAIYSTVASSLVLLVSLGAISFWMCSNGSLQCRICI

SEQ ID NO: 35 - protein, 266 aa, synthetic construct
  REGION 1..266: note = Construct derived from COVID-19 E484K_N501Y_K417N mutant (South African Mutant)
MFVFLVLLPLVSSVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTGVKLESMGIYQILAIYSTVASSLVLLVSLGAISFWMCSNGSLQCRICI

SEQ ID NO: 36 - protein, 266 aa, synthetic construct
  REGION 1..266: note = Construct derived from COVID-19 E484K mutant
MFVFLVLLPLVSSVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTNGVGYQPYRVVVLSFELLHAPATVCGPKKSTGVKLESMGIYQILAIYSTVASSLVLLVSLGAISFWMCSNGSLQCRICI

SEQ ID NO: 37 - protein, 207 aa, synthetic construct
  REGION 1..207: note = Fused protein comprising COVID-19 signal sequence (1-15) and COVID-19 RBD(330-521)
MFVFLVLLPLVSSQCPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAP

SEQ ID NO: 38 - protein, 260 aa, synthetic construct
  REGION 1..260: note = Fused protein comprising hIL-2 signal sequence, COVID-19 RBD(330-521)and HA(flexible-TM-Cyt)
MYRMQLLSCIALSLALVTNSPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPGVKLESMGIYQILAIYSTVASSLVLLVSLGAISFWMCSNGSLQCRICI

SEQ ID NO: 39 - protein, 364 aa, synthetic construct
  REGION 1..364: note = Fused protein comprising COVID-19 signal sequence (1-15), COVID-19 RBD(330-521), Human IgG4CH3 and Human CD80 (TM-Cyt)
MFVFLVLLPLVSSQCPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPGSGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGSLLPSWAITLISVNGIFVICCLTYCFAPRCRERRRNERLRRESVRPV

SEQ ID NO: 40 - protein, 366 aa, synthetic construct
  REGION 1..366: note = Fused protein comprising COVID-19 signal sequence (1-15), COVID-19 RBD(330-521), Human IgG4CH3 and HA(flexible-TM-Cyt)
MFVFLVLLPLVSSQCPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPGSGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGSGVKLESMGIYQILAIYSTVASSLVLLVSLGAISFWMCSNGSLQCRICI

SEQ ID NO: 41 - protein, 373 aa, synthetic construct
  REGION 1..373: note = Fused protein comprising COVID-19 signal sequence (1-15), COVID-19 RBD(330-530), Human IgG4CH3 and Human CD80 (TM-Cyt)
MFVFLVLLPLVSSQCPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSGSGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGSLLPSWAITLISVNGIFVICCLTYCFAPRCRERRRNERLRRESVRPV

SEQ ID NO: 42 - protein, 375 aa, synthetic construct
  REGION 1..375: note = Fused protein comprising COVID-19 signal sequence (1-15), COVID-19 RBD(330-530), Human IgG4CH3 and HA(flexible-TM-Cyt)
MFVFLVLLPLVSSQCPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSGSGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGSGVKLESMGIYQILAIYSTVASSLVLLVSLGAISFWMCSNGSLQCRICI

SEQ ID NO: 43 - protein, 369 aa, synthetic construct
  REGION 1..369: note = Fused protein comprising hIL-2 signal sequence, COVID-19 RBD(330-521), Human IgG4CH3 and Human CD80 (TM-Cyt)
MYRMQLLSCIALSLALVTNSPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPGSGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGSLLPSWAITLISVNGIFVICCLTYCFAPRCRERRRNERLRRESVRPV

SEQ ID NO: 44 - protein, 371 aa, synthetic construct
  REGION 1..371: note = Fused protein comprising hIL-2 signal sequence, COVID-19 RBD(330-521), Human IgG4CH3 and HA(flexible-TM-Cyt)
MYRMQLLSCIALSLALVTNSPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPGSGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGSGVKLESMGIYQILAIYSTVASSLVLLVSLGAISFWMCSNGSLQCRICI

SEQ ID NO: 45 - protein, 378 aa, synthetic construct
  REGION 1..378: note = Fused protein comprising hIL-2 signal sequence, COVID-19 RBD(330-530), Human IgG4CH3 and Human CD80 (TM-Cyt)
MYRMQLLSCIALSLALVTNSPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSGSGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGSLLPSWAITLISVNGIFVICCLTYCFAPRCRERRRNERLRRESVRPV

SEQ ID NO: 46 - protein, 380 aa, synthetic construct
  REGION 1..380: note = Fused protein comprising hIL-2 signal sequence, COVID-19 RBD(330-530), Human IgG4CH3 and HA(flexible-TM-Cyt)
MYRMQLLSCIALSLALVTNSPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSGSGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGSGVKLESMGIYQILAIYSTVASSLVLLVSLGAISFWMCSNGSLQCRICI

SEQ ID NO: 47 - protein, 266 aa, synthetic construct
  REGION 1..266: note = Fused protein comprising COVID-19 signal sequence (1-13), COVID-19 RBD(327-531) and HA(flexible-TM-Cyt)
MFVFLVLLPLVSSVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTGVKLESMGIYQILAIYSTVASSLVLLVSLGAISFWMCSNGSLQCRICI

SEQ ID NO: 48 - protein, 264 aa, synthetic construct
  REGION 1..264: note = Fused protein comprising COVID-19 signal sequence (1-15), COVID-19 RBD(330-521) and HA(flexible-TM-Cyt)
MFVFLVLLPLVSSQCPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSGVKLESMGIYQILAIYSTVASSLVLLVSLGAISFWMCSNGSLQCRICI

SEQ ID NO: 49 - protein, 291 aa, synthetic construct
  REGION 1..291: note = Fused protein comprising COVID-19 signal sequence (1-15), COVID-19 RBD(327-531) and COVID19 (JMD-TM)
MFVFLVLLPLVSSQCVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTCGKYEQYIKWPWYIWLGFIAGLIAIVMVTIMLCCMTSCCSCLKGCCSCGSCCKFDEDDSEPVLKGVKLHYT

SEQ ID NO: 50 - protein, 288 aa, synthetic construct
  REGION 1..288: note = Fused protein comprising COVID-19 signal sequence (1-13), COVID-19 RBD(327-531) and COVID19 (JMD-TM)
MFVFLVLLPLVSSVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTGKYEQYIKWPWYIWLGFIAGLIAIVMVTIMLCCMTSCCSCLKGCCSCGSCCKFDEDDSEPVLKGVKLHYT

SEQ ID NO: 51 - protein, 275 aa, synthetic construct
  REGION 1..275: note = Fused protein comprising COVID-19 signal sequence (1-13), COVID-19 RBD(330-521) and COVID19 (JMD-TM)
MFVFLVLLPLVSSPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPGKYEQYIKWPWYIWLGFIAGLIAIVMVTIMLCCMTSCCSCLKGCCSCGSCCKFDEDDSEPVLKGVKLHYT